(19) United States
(12) Patent Application Publication
(10) Pub. No.: US 2021/0311071 A1
(43) Pub. Date: Oct. 7, 2021

(54) Methods for Sample Quality Assessment

(71) Applicant: SOMALOGIC, INC., Boulder, CO (US)

(72) Inventors: Dominic Anthony ZICHI, Boulder, CO (US); Matthew Joel WESTACOTT, Thornton, CO (US)

(73) Assignee: SOMALOGIC, INC., Boulder, CO (US)

(21) Appl. No.: 17/287,959

(22) PCT Filed: Oct. 28, 2019
(86) PCT No.: PCT/US2019/058302

§ 371 Date: Apr. 22, 2021

Related U.S. Application Data
(60) Provisional application No. 62/752,947, filed on Oct. 30, 2018.

(51) Int. Cl. G01N 33/68

(52) U.S. Cl. CPC G01N 33/68

(57) ABSTRACT

The subject invention relates to methods for obtaining biological samples of improved quality. It encompasses the identification of markers or proteins in biological samples that are altered due to variations in sample collection, handling and processing. They are also useful for correcting variations in measured results for disease biomarkers. Further, they can permit the rejection of samples or groups of samples as necessary if it is determined that their collection method was not in accordance with the predetermined protocol. Other advantages useful to the skilled artisan are described herein.

### FIELD OF THE INVENTION

In the fields of medical diagnostics and drug development, comparisons are made between the composition of blood and other biological samples from individuals in order to determine and understand those changes which might be related to specific conditions or diseases. For example, biomarkers may indicate the ability to respond to certain medications, the presence of a disease such as cancer, or monitor processes such as the response to treatment or changes in organ function. Once established as reliable and robust, such biomarker measurements may be used clinically.

The key properties for an ideal biomarker measurement required for discovery as a biomarker and for further reaching clinical utility include reliability and robustness.

### BACKGROUND OF THE INVENTION

Blood contains powerful cellular and humoral systems for reacting to injury or foreign and infectious agents. Small challenges can induce the innate immune system (complement system and cells such as macrophages) to release powerful signals and enzymes, lead to activation of the platelets and trigger the coagulation of the blood. In as much as these signals are related to the processes inside the body, they are of interest because they can be directly involved in defense and repair systems and serve as markers for disease. However, such process signals are also responsive to the effects of blood sample preparation. Merely drawing blood from a vessel through a needle, or exposing blood to air can result in unintended activation of these mechanisms. For example, altering the time, centrifuge speed or temperature of sample processing steps can alter the apparent composition of serum or plasma such that physiologic information is masked by the pre-analytic variability imparted on the sample during collection and processing. The strong susceptibility of these processes and proteins to subtle alterations in sample handling of the proteins can compromise their use as biomarkers due to the concomitant lack of robustness.

Currently research efforts in multivariate biology show strong interest in pre-analytical sample variation (often called “batch effects”). Currently the extent to which sample quality can be determined is largely limited to visually obvious changes such as red color indicating red cell lysis, and cloudiness indicating high lipid or other contaminants. This limits the trust that clinicians can put in all but the hardiest and most robust protein measurements. A study documenting some of the complex and nonlinear effects of variations in serum and plasma preparation is described in Ostroff, R. et al. (2010) J. Proteomics 73:649-666. Proposed here are specific techniques that determine the compliance with sample preparation protocol, based on a nonlinear (logarithmic) transformation of measurements of a specific set of proteins affected by variation in sample preparation protocol. Metrics derived from these methods can be used to monitor compliance, reject samples, and make corrections in analytes of interest. These techniques are useful in evaluating the quality of human or animal blood samples used in biomarker research, clinical diagnostic applications, bio-bank sample quality monitoring and drug development. Similar approaches can be developed to assess sample integrity for many other sample types, including urine, cerebrospinal fluid, sputum or tissue.

### SUMMARY

As is described herein, the key properties for an ideal biomarker measurement required for biomarker discovery and for attaining clinical utility include reliability and robustness. Reliability of a biomarker means that the biomarker signal is truthful in capturing the underlying biology of health or disease (i.e., is not a “false positive” marker). Robustness of a biomarker indicates that the biomarkers are differentially expressed in diseased individuals relative to non-diseased individuals. To increase the probability of finding true disease biomarkers, and reduce the change of identifying false positives due to sample bias, a method for measuring sample quality and consistency is essential.

The measurement of protein analytes in plasma samples can be significantly affected by the protocol used to collect and handle the sample. Deviations from a specified sample collection and/or handling protocol can lead to changes in protein levels within the sample or other systematic effects on measurements that result in changes to signals for many analytes, including negative controls. Such deviations may occur irrespective of the type of assay used to measure the protein analytes.

In order to assess the quality of a set of clinical samples, the effects for the most obvious deviations from protocol have been characterized. Variability in protein composition as a function of time has been assessed between sample collection and spinning. Further, variability in protein composition as a function of time has been assessed between sample spinning and the time to decanting of the sample.

Signatures for sample mishandling have been identified that can be used as a quantitative classifier for assessing collections of clinical samples. Further, metrics have been produced for each analyte that capture the sensitivity of that analyte's measurements to deviations from collection protocol, particularly with respect to delay between sample collection and spinning and delays between sample spinning and sample decanting.

One might imagine that some techniques are relatively immune to the effects of sample handling, but this is not the case. Even though antibodies work well in the presence of blood plasma and serum matrices, and mass spectrometry can measure peptides and even denatured proteins, if cells in the samples lyse, or if platelets degranulate, or if the complement system is activated, then dramatic changes in analyte concentration will occur in the sample after it has been taken, and any “high fidelity” measurement technique will detect them. Therefore, techniques similar to those described herein for determination of the impact of sample handling variations can be useful for multiple assay formats and biomarkers other than proteins. Such assay formats may be sensitive in different ways, but can be affected by the same underlying causes in terms of sample preparation variation.

The variations of the different steps in blood handling and processing can be shown to affect biological samples in reproducible ways. The sensitivity of each biomarker protein measurement to parameters associated with the various sample handling and processing steps have been quantified using the SOMAmer® proteomic array and markers of variation in sample handling processes have been identified. The sample handling and processing variations have been quantified within the same multianalyte measurement assay for disease biomarker measurements and for developed methods, to determine which handling/processing markers have been affected, and approximately by how much. The subject methods have also made it possible to place limits on acceptable sample handling and processing quality metrics for biomarker discovery.

The following numbered paragraphs describe further aspects of the present invention:

1. A method comprising:
a) measuring the level of Sonic Hedgehog (SHH) protein, and the level of at least one, two, three, four, five, six, seven, eight, nine, ten, eleven, twelve or thirteen proteins selected from the group consisting of PGAM1, PGAM2, C4A.C4B, PTPN4, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

2. The method of claim 1, wherein the measuring is performed using mass spectrometry, an aptamer based assay and/or an antibody based assay.

3. The method of claim 1, wherein the sample is selected from blood, plasma, serum or urine.

4. The method of claim 1, wherein the method comprises measuring SHH and PGAM1, SHH and PTPN4, SHH and TNFSF14, SHH and FAM49B, SHH and RBP7, SHH and IHH, SHH and DDX39B, SHH and S100A12, SHH and PGAM2, SHH and C4A.C4B, SHH and IL21R, SHH and TMEM9 or SHH and ADAM9.

5. The method of claim 1, wherein the method comprises measuring SHH, PGAM1 and TNFSF14; SHH, PGAM1 and RBB7; SHH, PGAM1 and PTPN4; SHH, PGAM1 and DDX39B; SHH, PGAM1 and FAM49B; SHH, PGAM1 and IHH; SHH, PGAM1 and S100A12; SHH, PGAM1 and ADAM9; SHH, PTPN4 and RBP7; SHH, PTPN4 and TNFSF14; SHH, PTPN4 and IHH; SHH, RBP7 and FAM49B; SHH, RBP7 AND IHH; SHH, FAM49B and TNFSF14; SHH, DDX39B and PTPN4; SHH, TNFSF14 and S100A12; SHH, IHH and RBP7; SHH, IHH and TNFSF14; SHH, RBP7 and TNFSF14; SHH, RBP7 and S100A12; SHH, RBP7 and DDX39B; SHH, TNFSF14 and DDX39B; SHH, S100A12 and DDX39B; SHH, FAM49B and S100A12; SHH, IHH and FAM49B; SHH, IHH and DDX39B; SHH, TNFSF14 and ADAM9; SHH FAM49B and DDX39B; SHH, IHH and ADAM9; SHH, PGAM1 and C4A.C4B; SHH, PGAM2 and RBP7; SHH, PGAM1 and IL21R; SHH, PGAM2 and PTPN4; SHH, PGAM2 and ADAM9, SHH, PGAM2 and C4A.C4B; SHH, PGAM2 and IL21R; SHH, IHH and PGAM2; SHH, PGAM1 and PGAM2; SHH, TMEM9 and PGAM2 or SHH, TMEM9 and PGAM1.

6. The method of claim 1, wherein the method comprises measuring SHH and PGAM1, and at least two of the following proteins selected from RBP7, TNFSF14, PTPN4, DDX39B, FAM49B, S100A12, IHH, PGAM2, C4A.C4B, IL21R, TMEM9 and ADAM9.

7. The method of claim 1, wherein the method comprises measuring SHH and IHH, and at least two of the following proteins selected from RBP7, TNFSF14, PTPN4, DDX39B, FAM49B, S100A12, PGAM1, PGAM2, C4A.C4B, IL21R, TMEM9 and ADAM9.

8. The method of anyone of the preceding claims, wherein the protein levels are used to predict the length of time between the sample collection from the human subject and sample centrifugation and/or the length of time between sample centrifugation and sample decanting.

9. The method of claim 8, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

10. A method comprising:
a) contacting a sample from a human subject with a set of capture reagents, wherein each capture reagent has affinity for a different protein of the set of proteins comprising Sonic Hedgehog (SHH), and at least one, two, three, four, five, six, seven, eight, nine, ten, eleven, twelve or thirteen proteins selected from the group consisting of PGAM1, PTPN4, TNFSF14, FAM49B, RBP7, IHH, DDX39B, S100A12, ADAM9, PGAM2, C4A.C4B, IL21R and TMEM9; and
b) measuring the level of each protein of the set of proteins based with the set of capture reagents.

11. The method of claim 10, wherein the set of capture reagents are selected from aptamers, antibodies and a combinations of aptamers and antibodies.

12. The method of claim 10, wherein the sample is selected from blood, plasma, serum or urine.

13. The method of claim 10, wherein the method comprises measuring SHH and PGAM1, SHH and PTPN4, SHH and TNFSF14, SHH and FAM49B, SHH and RBP7, SHH and IHH, SHH and DDX39B, SHH and S100A12, SHH and PGAM2, SHH and C4A.C4B, SHH and IL21R, SHH and TMEM9 or SHH and ADAM9.

14. The method of claim 10, wherein the method comprises measuring SHH, PGAM1 and TNFSF14; SHH, PGAM1 and RBB7; SHH, PGAM1 and PTPN4; SHH, PGAM1 and DDX39B; SHH, PGAM1 and FAM49B; SHH, PGAM1 and IHH; SHH, PGAM1 and S100A12; SHH, PGAM1 and ADAM9; SHH, PTPN4 and RBP7; SHH, PTPN4 and TNFSF14; SHH, PTPN4 and IHH; SHH, RBP7 and FAM49B; SHH, RBP7 AND IHH; SHH, FAM49B and TNFSF14; SHH, DDX39B and PTPN4; SHH, TNFSF14 and S100A12; SHH, IHH and RBP7; SHH, IHH and TNFSF14; SHH RBP7 and TNFSF14; SHH, RBP7 and S100A12; SHH, RBP7 and DDX39B; SHH, TNFSF14 and DDX39B; SHH, S100A12 and DDX39B; SHH, FAM49B and S100A12; SHH, IHH and FAM49B; SHH, IHH and DDX39B; SHH, TNFSF14 and ADAM9; SHH FAM49B and DDX39B; SHH, IHH and ADAM9; SHH, PGAM1 and C4A.C4B; SHH, PGAM2 and RBP7; SHH, PGAM1 and IL21R; SHH, PGAM2 and PTPN4; SHH, PGAM2 and ADAM9, SHH, PGAM2 and C4A.C4B; SHH, PGAM2 and IL21R; SHH, IHH and PGAM2; SHH, PGAM1 and PGAM2; SHH, TMEM9 and PGAM2 or SHH, TMEM9 and PGAM1.

15. The method of claim 10, wherein the method comprises measuring SHH and PGAM1, and at least two of the following proteins selected from RBP7, TNFSF14, PTPN4, DDX39B, FAM49B, S100A12, IHH and ADAM9.

16. The method of claim 10, wherein the method comprises measuring SHH and IHH, and at least two of the following proteins selected from RBP7, TNFSF14, PTPN4, DDX39B, FAM49B, S100A12, PGAM1 and ADAM9.

17. The method of anyone of the preceding claims, wherein the protein levels are used to predict the length of time between the sample collection from the human subject and sample centrifugation and/or the length of time between sample centrifugation and sample decanting.

18. The method of claim 17, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

19. A method comprising:
a) measuring the level of at least three, four, five, six, seven or eight proteins selected from the group consisting of IHH, PTPN4, TNFSF14, FAM49B, RBP7, DDX39B, S100A12, and ADAM9 in a sample from a subject; and
b) identifying the sample as an analysis sample or negative sample based on the level of the three, four, five, six, seven or eight proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

20. The method of claim 19, wherein the measuring is performed using mass spectrometry, an aptamer based assay and/or an antibody based assay.

21. The method of claim 19, wherein the sample is selected from blood, plasma, serum or urine.

22. The method of claim 19, wherein the method comprises measuring IHH, RB7 and PTPN4; IHH, RB7 and TNFSF14; IHH, RB7 and FAM49B; IHH, RBP7 and DDX39B; IHH, RBP7 and S100A12; IHH, RB7 and ADAM9; IHH, TNFSF14 and PTPN4; IHH, TNFSF14 and FAM49B; IHH, TNFSF14 and DDX39B; IHH, TNFSF14 and S100A12; IHH, TNFSF14 and ADAM9; IHH, FAM49 and PTPN4; IHH, FAM49 and TNFSF14; IHH, FAM49 and DDX39B; IHH, FAM49 and S100A12; IHH, ADAM9 and PTPN4 or IHH, FAM49 and ADAM9.

23. The method of anyone of the preceding claims, further comprising measuring SHH and/or PGAM1.

24. The method of anyone of the preceding claims, wherein the protein levels are used to predict the length of time between the sample collection from the human subject and sample centrifugation and/or the length of time between sample centrifugation and sample decanting.

25. The method of claim 19, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

26. A method comprising:
a) contacting a sample from a human subject with a set of capture reagents, wherein each capture reagent has affinity for a different protein of the set of proteins comprising three, four, five, six, seven or eight proteins selected from the group consisting of IHH, PTPN4, TNFSF14, FAM49B, RBP7, DDX39B, S100A12, and ADAM9 in a sample from a subject; and
b) measuring the level of each protein of the set of proteins with the set of capture reagents.

27. The method of claim 26, wherein the set of capture reagents are selected from aptamers, antibodies and a combinations of aptamers and antibodies.

28. The method of claim 26, wherein the sample is selected from blood, plasma, serum or urine.

29. The method of claim 26, wherein the method comprises measuring IHH, RB7 and PTPN4; IHH, RB7 and TNFSF14; IHH, RB7 and FAM49B; IHH, RBP7 and DDX39B; IHH, RBP7 and S100A12; IHH, RB7 and ADAM9; IHH, TNFSF14 and PTPN4; IHH, TNFSF14 and FAM49B; IHH, TNFSF14 and DDX39B; IHH, TNFSF14 and S100A12; IHH, TNFSF14 and ADAM9; IHH, FAM49 and PTPN4; IHH, FAM49 and TNFSF14; IHH, FAM49 and DDX39B; IHH, FAM49 and S100A12; or IHH, FAM49 and ADAM9.

30. The method of anyone of the preceding claims, further comprising measuring SHH and/or PGAM1.

31. The method of anyone of the preceding claims, wherein the protein levels are used to predict the length of time between the sample collection from the human subject and sample centrifugation and/or the length of time between sample centrifugation and sample decanting.

32. The method of claim 26, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

33. A method comprising:
a) measuring the level of RB7, FAM49B, TNFSF14, ADAM9, PGAM1 and at least one protein selected from DDX39B and S100A12 in a sample from a human subject; and
b) identifying the sample as an analysis sample or negative sample based on the level of RB7, FAM49B, TNFSF14, ADAM9, PGAM1 and the at least one protein;
wherein, the analysis sample is a sample that is used in one or more of the following: protein biomarker discovery analysis, protein expression level analysis, a diagnostic method or a prognostic method, and the negative sample is a sample that is not used as an analysis sample.

34. The method of claim 33, wherein the measuring is performed using mass spectrometry, an aptamer based assay and/or an antibody based assay.

35. The method of claim 33, wherein the sample is selected from blood, plasma, serum or urine.

36. The method of claim 33, wherein the method comprises measuring RB7, FAM49B, TNFSF14, ADAM9, PGAM1 and S100A12; or RB7, FAM49B, TNFSF14, ADAM9, PGAM1 and DDX39B.

37. The method of anyone of the preceding claims, wherein the protein levels are used to predict the length of time between the sample collection from the human subject and sample centrifugation and/or the length of time between sample centrifugation and sample decanting.

38. The method of claim 33, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

39. A method comprising:
a) contacting a sample from a human subject with a set of capture reagents, wherein each capture reagent has affinity for a different protein of the set of proteins comprising RB7, FAM49B, TNFSF14, ADAM9, PGAM1 and at least one protein selected from DDX39B and S100A12; and
b) measuring the level of each protein of the set of proteins based with the set of capture reagents.

40. The method of claim 39, wherein the set of capture reagents are selected from aptamers, antibodies and a combinations of aptamers and antibodies.

41. The method of claim 39, wherein the sample is selected from blood, plasma, serum or urine.

42. The method of claim 39, wherein the method comprises measuring RB7, FAM49B, TNFSF14, ADAM9, PGAM1 and S100A12; or RB7, FAM49B, TNFSF14, ADAM9, PGAM1 and DDX39B.

43. The method of anyone of the preceding claims, wherein the protein levels are used to predict the length of time between the sample collection from the human subject and sample centrifugation and/or the length of time between sample centrifugation and sample decanting.

44. The method of claim 39, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

45. The method of anyone of the preceding claims, wherein the protein level or levels are used to assign a quality score to the sample, wherein the quality score is then used to determine whether the sample is an analysis sample or a non-analysis sample.

46. The method of anyone of the preceding claims, wherein the protein level or levels are used to assign a quality score to the sample, wherein the quality score is then used to determine if the sample is used for further analysis of additional proteins in the sample.

47. A method comprising:
a) measuring the level of PGAM1 protein, and the level of at least one, two, three, four, five, six, seven, eight, nine, ten, eleven, twelve or thirteen proteins selected from the group consisting of SHH, PGAM2, C4A.C4B, PTPN4, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

48. A method comprising:
a) measuring the level of PGAM2 protein, and the level of at least one, two, three, four, five, six, seven, eight, nine, ten, eleven, twelve or thirteen proteins selected from the group consisting of SHH, PGAM1, C4A.C4B, PTPN4, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

49. A method comprising:
a) measuring the level of C4A.C4B protein, and the level of at least one, two, three, four, five, six, seven, eight, nine, ten, eleven, twelve or thirteen proteins selected from the group consisting of SHH, PGAM2, PGAM1, PTPN4, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

50. A method comprising:
a) measuring the level of PTPN4 protein, and the level of at least one, two, three, four, five, six, seven, eight, nine, ten, eleven, twelve or thirteen proteins selected from the group consisting of SHH, PGAM2, C4A.C4B, PGAM1, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.\

51. A method comprising:
a) measuring the level of TNFSF14 protein, and the level of at least one, two, three, four, five, six, seven, eight, nine, ten, eleven, twelve or thirteen proteins selected from the group consisting of SHH, PGAM2, C4A.C4B, PTPN4, PGAM1, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

52. A method comprising:
a) measuring the level of FAM49B protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

53. A method comprising:
a) measuring the level of RBP7 protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

54. A method comprising:
a) measuring the level of IHH protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, RBP7, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

55. A method comprising:
a) measuring the level of DDX39B protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, RBP7, IHH, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

56. A method comprising:
a) measuring the level of S100A12 protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, RBP7, IHH, DDX39B, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

57. A method comprising:
a) measuring the level of IL21R protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, RBP7, IHH, S100A12, DDX39B, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

58. A method comprising:
a) measuring the level of TMEM9 protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, RBP7, IHH, S100A12, IL21R, DDX39B and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

59. A method comprising:
a) measuring the level of ADAM9 protein, and the level of at least one, two, three, four, five, six, seven, eight, nine, ten, eleven, twelve or thirteen proteins selected from the group consisting of SHH, PGAM2, C4A.C4B, PTPN4, TNFSF14, PGAM1, FAM49B, RBP7, IHH, S100A12, IL21R, TMEM9 and DDX39B in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

60. A method comprising:
a) contacting a sample from a human subject with a set of capture reagents, wherein each capture reagent has affinity for a different protein of the set of proteins comprising Sonic Hedgehog (SHH) protein, and the level of at least one, two, three, four, five, six, seven, eight, nine, ten, eleven, twelve or thirteen proteins selected from the group consisting of PGAM1, PGAM2, C4A.C4B, PTPN4, TNFSF14, FAM49B, RBP7, IHH, DDX39B, S100A12, IL21R, TMEM9 and ADAM9; and
b) measuring the level of each protein of the set of proteins based with the set of capture reagents.

61. A method comprising:
a) contacting a sample from a human subject with a set of capture reagents, wherein each capture reagent has affinity for a different protein of the set of proteins comprising PGAM1 protein, and the level of at least one, two, three, four, five, six, seven, eight, nine, ten, eleven, twelve or thirteen proteins selected from the group consisting of SHH, PGAM2, C4A.C4B, PTPN4, TNFSF14, FAM49B, RBP7, IHH, DDX39B, S100A12, IL21R, TMEM9 and ADAM9; and
b) measuring the level of each protein of the set of proteins based with the set of capture reagents.

62. A method comprising:
a) contacting a sample from a human subject with a set of capture reagents, wherein each capture reagent has affinity for a different protein of the set of proteins comprising PGAM2 protein, and the level of at least one, two, three, four, five, six, seven, eight, nine, ten, eleven, twelve or thirteen proteins selected from the group consisting of SHH, PGAM1, C4A.C4B, PTPN4, TNFSF14, FAM49B, RBP7, IHH, DDX39B, S100A12, IL21R, TMEM9 and ADAM9; and
b) measuring the level of each protein of the set of proteins based with the set of capture reagents.

63. A method comprising:
a) contacting a sample from a human subject with a set of capture reagents, wherein each capture reagent has affinity for a different protein of the set of proteins comprising C4A.C4B protein, and the level of at least one, two, three, four, five, six, seven, eight, nine, ten, eleven, twelve or thirteen proteins selected from the group consisting of SHH, PGAM1, PGAM2, PTPN4, TNFSF14, FAM49B, RBP7, IHH, DDX39B, S100A12, IL21R, TMEM9 and ADAM9; and
b) measuring the level of each protein of the set of proteins based with the set of capture reagents.

64. A method comprising:
a) contacting a sample from a human subject with a set of capture reagents, wherein each capture reagent has affinity for a different protein of the set of proteins comprising PTPN4 protein, and the level of at least one, two, three, four, five, six, seven, eight, nine, ten, eleven, twelve or thirteen proteins selected from the group consisting of SHH, PGAM1, PGAM2, C4A.C4B, TNFSF14, FAM49B, RBP7, IHH, DDX39B, S100A12, IL21R, TMEM9 and ADAM9; and
b) measuring the level of each protein of the set of proteins based with the set of capture reagents.

65. A method comprising:
a) contacting a sample from a human subject with a set of capture reagents, wherein each capture reagent has affinity for a different protein of the set of proteins comprising TNFSF14 protein, and the level of at least one, two, three, four, five, six, seven, eight, nine, ten, eleven, twelve or thirteen proteins selected from the group consisting of SHH, PGAM1, PGAM2, C4A.C4B, PTPN4, FAM49B, RBP7, IHH, DDX39B, S100A12, IL21R, TMEM9 and ADAM9; and
b) measuring the level of each protein of the set of proteins based with the set of capture reagents.

66. A method comprising:
a) contacting a sample from a human subject with a set of capture reagents, wherein each capture reagent has affinity for a different protein of the set of proteins comprising FAM49B protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, RBP7, IHH, DDX39B, S100A12, IL21R, TMEM9 and ADAM9; and
b) measuring the level of each protein of the set of proteins based with the set of capture reagents.

67. A method comprising:
a) contacting a sample from a human subject with a set of capture reagents, wherein each capture reagent has affinity for a different protein of the set of proteins comprising RBP7 protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, IHH, DDX39B, S100A12, IL21R, TMEM9 and ADAM9; and
b) measuring the level of each protein of the set of proteins based with the set of capture reagents.

68. A method comprising:
a) contacting a sample from a human subject with a set of capture reagents, wherein each capture reagent has affinity for a different protein of the set of proteins comprising IHH protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, RBP7, DDX39B, S100A12, IL21R, TMEM9 and ADAM9; and
b) measuring the level of each protein of the set of proteins based with the set of capture reagents.

69. A method comprising:
a) contacting a sample from a human subject with a set of capture reagents, wherein each capture reagent has affinity for a different protein of the set of proteins comprising DDX39B protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, RBP7, IHH, S100A12, IL21R, TMEM9 and ADAM9; and
b) measuring the level of each protein of the set of proteins based with the set of capture reagents.

70. A method comprising:
a) contacting a sample from a human subject with a set of capture reagents, wherein each capture reagent has affinity for a different protein of the set of proteins comprising S100A12 protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, RBP7, IHH, DDX39B, IL21R, TMEM9 and ADAM9; and
b) measuring the level of each protein of the set of proteins based with the set of capture reagents.

71. A method comprising:
a) contacting a sample from a human subject with a set of capture reagents, wherein each capture reagent has affinity for a different protein of the set of proteins comprising IL21R protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, RBP7, IHH, DDX39B, S100A12, TMEM9 and ADAM9; and
b) measuring the level of each protein of the set of proteins based with the set of capture reagents.

72. A method comprising:
a) contacting a sample from a human subject with a set of capture reagents, wherein each capture reagent has affinity for a different protein of the set of proteins comprising TMEM9 protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, RBP7, IHH, DDX39B, S100A12, IL21R and ADAM9; and
b) measuring the level of each protein of the set of proteins based with the set of capture reagents.

73. A method comprising:
a) contacting a sample from a human subject with a set of capture reagents, wherein each capture reagent has affinity for a different protein of the set of proteins comprising ADAM9 protein, and the level of at least one, two, three, four, five, six, seven, eight, nine, ten, eleven, twelve or thirteen proteins selected from the group consisting of SHH, PGAM1, PGAM2, C4A.C4B, PTPN4, TNFSF14, FAM49B, RBP7, IHH, DDX39B, S100A12, IL21R and TMEM9; and
b) measuring the level of each protein of the set of proteins based with the set of capture reagents.

74. A method comprising:
a) measuring the level of Sonic Hedgehog (SHH) protein, and the level of at least one, two, three, four, five, six, seven, eight, nine, ten, eleven, twelve or thirteen proteins selected from the group consisting of PGAM1, PGAM2, C4A.C4B, PTPN4, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

75. A method comprising:
a) measuring the level of HNRNDPDL, PTPN4, PGAM2, C4A.C4B, EIF4A1, IHH, SHH, PGAM1, S100A9 and HLA.DRB3 in a sample from a human subject; and
b) identifying the sample as an analysis sample or negative sample based on the level of HNRNDPDL, PTPN4, PGAM2, C4A.C4B, EIF4A1, IHH, SHH, PGAM1, S100A9 and HLA.DRB3;
wherein, the analysis sample is a sample that is used in one or more of the following: protein biomarker discovery analysis, protein expression level analysis, a diagnostic method or a prognostic method, and the negative sample is a sample that is not used as an analysis sample.

76. The method of anyone of the preceding claims, wherein the measuring is performed using mass spectrometry, an aptamer based assay and/or an antibody based assay.

77. The method of anyone of the preceding claims, wherein the sample is selected from blood, plasma, serum or urine.

78. The method of anyone of the preceding claims, wherein the protein levels are used to predict the length of time between the sample collection from the human subject and sample centrifugation and/or the length of time between sample centrifugation and sample decanting.

79. The method of anyone of the preceding claims wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

80. A method comprising:
a) contacting a sample from a human subject with a set of capture reagents, wherein each capture reagent has affinity for a different protein of the set of proteins comprising HNRNDPDL, PTPN4, PGAM2, C4A.C4B, EIF4A1, IHH, SHH, PGAM1, S100A9 and HLA.DRB3; and
b) measuring the level of each protein of the set of proteins based with the set of capture reagents.

81. The method of anyone of the preceding claims, wherein the measuring is performed using mass spectrometry, an aptamer based assay and/or an antibody based assay.

82. The method of anyone of the preceding claims, wherein the sample is selected from blood, plasma, serum or urine.

83. The method of anyone of the preceding claims, wherein the protein levels are used to predict the length of time between the sample collection from the human subject and sample centrifugation and/or the length of time between sample centrifugation and sample decanting.

84. The method of anyone of the preceding claims wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

85. A method comprising:
a) contacting a sample from a human subject with two capture reagents, wherein one capture reagent has affinity for a TMEM9 protein and the second capture reagent has affinity for a PGAM1 protein; and
b) measuring the level of each protein with the two capture reagents.

86. A method comprising:
a) measuring the level of PGAM1 and TMEM9 proteins in a sample from a human subject; and
b) identifying the sample as an analysis sample or negative sample based on the level of PGAM1 and TMEM9;
wherein, the analysis sample is a sample that is used in one or more of the following: protein biomarker discovery analysis, protein expression level analysis, a diagnostic method or a prognostic method, and the negative sample is a sample that is not used as an analysis sample.

87. The method of claim 85 or 86 further comprising measuring the level of an IHH protein with a capture reagent having affinity for the IHH protein.

88. The method of claim 85 or 86 further comprising measuring the level of an C4A.C4B protein with a capture reagent having affinity for the C4A.C4B protein.

89. The method of claim 85 or 86 further comprising measuring the level of an SHH protein with a capture reagent having affinity for the SHH protein.

90. The method of claim 85 or 86 further comprising measuring the level of a PGAM2 protein with a capture reagent having affinity for the PGAM2 protein.

91. The method of claim 85 or 86 further comprising measuring the level of an ADAM9 protein with a capture reagent having affinity for the ADAM9 protein.

92. The method of claim 85 or 86 further comprising measuring the level of a PTPN4 protein with a capture reagent having affinity for the PTPN4 protein.

93. The method of claim 84 or 85 further comprising measuring the level of an IL21R protein with a capture reagent having affinity for the IL21R protein.

94. The method of claim 85 or 86 further comprising measuring the level of an RBP7 protein with a capture reagent having affinity for the RBP7 protein.

95. A method comprising:
a) contacting a sample from a human subject with two capture reagents, wherein one capture reagent has affinity for a SHH protein and the second capture reagent has affinity for a IHH protein; and
b) measuring the level of each protein with the two capture reagents.

96. A method comprising:
a) measuring the level of SHH and IHH in a sample from a human subject; and
b) identifying the sample as an analysis sample or negative sample based on the level of SHH and IHH;
wherein, the analysis sample is a sample that is used in one or more of the following: protein biomarker discovery analysis, protein expression level analysis, a diagnostic method or a prognostic method, and the negative sample is a sample that is not used as an analysis sample.

97. The method of claim 95 or 96 further comprising measuring the level of an RBP7 protein with a capture reagent having affinity for the RBP7 protein.

98. The method of claim 95 or 96 further comprising measuring the level of an FAM94B protein with a capture reagent having affinity for the FAM94B protein.

99. The method of claim 95 or 96 further comprising measuring the level of an TNFSF14 protein with a capture reagent having affinity for the TNFS14 protein

100. The method of claim 95 or 96 further comprising measuring the level of an ADAM9 protein with a capture reagent having affinity for the ADAM9 protein.

101. The method of claim 95 or 96 further comprising measuring the level of an S100A12 protein with a capture reagent having affinity for the S100A12 protein.

102. The method of claim 95 or 96 further comprising measuring the level of an DDX39B protein with a capture reagent having affinity for the DDX39B protein.

103. The method of claim 95 or 96 further comprising measuring the level of an PGAM1 protein with a capture reagent having affinity for the PGAM1 protein.

104. The method of claim 95 or 96 further comprising measuring the level of a PTPN4 protein with a capture reagent having affinity for the PTPN4 protein.

105. A method comprising:
a) contacting a sample from a human subject with four capture reagents, wherein each of the four capture reagents has affinity for a protein selected from IHH, RBP7, ADAM9 and PTPN4; and
b) measuring the level of each protein with the four capture reagents.

106. The method of claim 105 further comprising measuring the level of a SHH protein with a capture reagent having affinity for the SHH protein.

107. The method of claim 105 further comprising measuring the level of a PGAM1 protein with a capture reagent having affinity for the PGAM1 protein.

108. The method of claim 105 further comprising measuring the level of one or more proteins selected from TMEM9, C4A.C4B, PGAM2, FAM49B, TNFSF14, S100A12, DDX39B and IL21R with capture reagents, each capture reagent having affinity for one of the one or more proteins.

109. A method comprising:
a) measuring the level of IHH, RBP7, ADAM9 and PTPN4 proteins in a sample from a human subject; and
b) identifying the sample as an analysis sample or negative sample based on the level of IHH, RBP7, ADAM9 and PTPN4 proteins from the sample;
wherein, the analysis sample is a sample that is used in one or more of the following: protein biomarker discovery analysis, protein expression level analysis, a diagnostic method or a prognostic method, and the negative sample is a sample that is not used as an analysis sample.

110. The method of 109 further comprising measuring the level of a SHH protein and identifying the sample as an analysis sample or negative sample based on the level of the SHH protein from the sample.

111. The method of 109 further comprising measuring the level of a PGAM1 protein and identifying the sample as an analysis sample or negative sample based on the level of the PGAM1 protein from the sample.

112. The method of claim 109 further comprising measuring the level of one or more proteins selected from TMEM9, C4A.C4B, PGAM2, FAM49B, TNFSF14, S100A12, DDX39B and IL21R and identifying the sample as an analysis sample or negative sample based on the level of the one or more proteins.

113. A method comprising:
a) contacting a sample from a human subject with four capture reagents, wherein each of the four capture reagents has affinity for a protein selected from IHH, RBP7, ADAM9 and PTPN4; and
b) measuring the level of each protein in the sample with the four capture reagents.

114. The method of claim 113 further comprising measuring the level of a SHH protein with a capture reagent having affinity for the SHH protein.

115. The method of claim 113 further comprising measuring the level of a PGAM1 protein with a capture reagent having affinity for the PGAM1 protein.

116. The method of claim 113 further comprising measuring the level of a TMEM9 protein with a capture reagent having affinity for the TMEM9 protein.

117. The method of claim 113 further comprising measuring the level of one or more proteins selected from C4A.C4B, PGAM2, FAM49B, TNFSF14, S100A12, DDX39B and IL21R with capture reagents, each capture reagent having affinity for one of the one or more proteins.

118. The method of claim 113, wherein the sample is selected from blood, plasma, serum or urine.

119. The method of claim 113, wherein the protein levels are used to predict the length of time between the sample collection from the human subject and sample centrifugation and/or the length of time between sample centrifugation and sample decanting.

120. The method of claim 113, wherein the protein levels are used to identify the sample as an analysis sample or negative sample based on the level of the proteins; wherein, the analysis sample is a sample that is used in one or more of the following: protein biomarker discovery analysis, protein expression level analysis, a diagnostic method or a prognostic method, and the negative sample is a sample that is not used as an analysis sample.

121. The method of claim 119, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

122. The method of claim 113, wherein the capture reagents are selected from an aptamer or an antibody.

123. A method comprising:
a) measuring the level of IHH, RBP7, ADAM9 and PTPN4 proteins in a sample from a human subject; and
b) identifying the sample as an analysis sample or negative sample based on the level of IHH, RBP7, ADAM9 and PTPN4 proteins from the sample;
wherein, the analysis sample is a sample that is used in one or more of the following: protein biomarker discovery analysis, protein expression level analysis, a diagnostic method or a prognostic method, and the negative sample is a sample that is not used as an analysis sample.

124. The method of 123 further comprising measuring the level of a SHH protein and identifying the sample as an analysis sample or negative sample based on the level of the SHH protein from the sample.

125. The method of 123 further comprising measuring the level of a PGAM1 protein and identifying the sample as an analysis sample or negative sample based on the level of the PGAM1 protein from the sample.

126. The method of 123 further comprising measuring the level of a TMEM9 protein and identifying the sample as an analysis sample or negative sample based on the level of the TMEM9 protein from the sample.

127. The method of claim 123 further comprising measuring the level of one or more proteins selected from C4A.C4B, PGAM2, FAM49B, TNFSF14, S100A12, DDX39B and IL21R and identifying the sample as an analysis sample or negative sample based on the level of the one or more proteins.

128. The method of claim 123, wherein the sample is selected from blood, plasma, serum or urine.

129. The method of claim 123, wherein the protein levels are used to predict the length of time between the sample collection from the human subject and sample centrifugation and/or the length of time between sample centrifugation and sample decanting.

130. The method of claim 129, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

131. The method of claim 123, wherein the measuring of the protein levels is performed using mass spectrometry, an aptamer based assay and/or an antibody based assay.

132. The method of claim 123, wherein the protein levels are used in a classifier selected from a decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; random forest model is used with the protein levels to identify a sample as an analysis sample or a negative sample.

### BRIEF DESCRIPTION OF THE DRAWINGS

FIG. 1 illustrates PGAM1 RFU vs. Time-To-Spin which shows robust change in signal with time-to-spin.

FIG. 2 illustrates Analyte RFU vs. Time-To-Spin which shows very low discriminatory properties.

FIG. 3 illustrates analyte importance in time-to-spin model. After ˜10 analytes the relative importance of additional analytes decreases to a steady state.

FIG. 4 illustrates a sample decision tree in the time-to-spin model. The first node splits a sample on TNFSF14 RFU, it either terminates with a prediction of 24 hours if the RFU is greater than 756.2 or traverses down additional branches otherwise.

FIG. 5 illustrates error in prediction vs. number of trees in random forest.

FIG. 6 illustrates prediction errors in a single analyte random forest model. Horizontal and vertical bars indicate class thresholding and solid black line indicates the true prediction line.

FIG. 7 illustrates model stability in random forest and Naive Bayes. Whereas the Naive Bayes model shows continuous change when shifting the signal on a single analyte the random forest shows more stability.

FIG. 8 illustrates model stability when scaling individual analytes. The true time-to-spin given on each panel title is compared against the prediction time when scaling each analyte by an effect size. Individual lines represent the prediction of the random forest when scaling that analyte and leaving the remaining nine constant.

FIG. 9 illustrates cumulative analyte distribution functions for 18 individuals with varying time-to-spin for protein marker SHH.

FIG. 10 illustrates cumulative analyte distribution functions for 18 individuals with varying time-to-spin for protein marker IHH.

FIG. 11 illustrates cumulative analyte distribution functions for 18 individuals with varying time-to-spin for protein marker RBP7.

FIG. 12 illustrates cumulative analyte distribution functions for 18 individuals with varying time-to-spin for protein marker FAM49B.

FIG. 13 illustrates cumulative analyte distribution functions for 18 individuals with varying time-to-spin for protein marker TNFSF14.

FIG. 14 illustrates cumulative analyte distribution functions for 18 individuals with varying time-to-spin for protein marker ADAM9.

FIG. 15 illustrates cumulative analyte distribution functions for 18 individuals with varying time-to-spin for protein marker S100A12.

FIG. 16 illustrates cumulative analyte distribution functions for 18 individuals with varying time-to-spin for protein marker DDX39B.

FIG. 17 illustrates cumulative analyte distribution functions for 18 individuals with varying time-to-spin for protein marker PGAM1.

FIG. 18 illustrates cumulative analyte distribution functions for 18 individuals with varying time-to-spin for protein marker PTPN4.

FIG. 19 illustrates the performance of analyte models wherein the performance of each model was quantified using the RMSE for the predicted time-to-spin against the true time-to-spin for each individual and timepoint.

FIG. 20 illustrates low performance analytes based on the fraction of times an analyte was used in each grouping of model performance to elucidate the importance of each analyte on model performance.

FIG. 21 illustrates mid performance analytes based on the fraction of times an analyte was used in each grouping of model performance to elucidate the importance of each analyte on model performance.

FIG. 22 illustrates high performance analytes based on the fraction of times an analyte was used in each grouping of model performance to elucidate the importance of each analyte on model performance.

FIG. 23 illustrates the distribution of the number of models used with the specified number of analytes.

### DESCRIPTION OF THE INVENTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “an aptamer” includes mixtures of aptamers, reference to “a probe” includes mixtures of probes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

As used herein, “biomarker” is used to refer to a target molecule that indicates or is a sign of a normal or abnormal process in an individual or of a disease or other condition in an individual. More specifically, a “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker.

Biomarker selection for a specific disease state involves first the identification of markers that have a measurable and statistically significant difference in a disease population compared to a control population for a specific medical application. Biomarkers can include secreted or shed molecules that parallel disease development or progression and readily diffuse into the bloodstream from tissue affected by a disease or condition or from surrounding tissues and circulating cells in response to a disease or condition. The biomarker or set of biomarkers identified are generally clinically validated or shown to be a reliable indicator for the original intended use for which it was selected. Biomarkers can comprise a variety of molecules including small molecules, peptides, proteins, and nucleic acids. Some of the key issues that affect the identification of biomarkers include over-fitting of the available data and bias in the data including sample handling protocol variations.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

“Disease biomarker control range” or “biomarker control range” are used interchangeably and mean the normal or non-disease range of biomarkers in non-diseased or normal individuals. They are typically derived from a control population.

“Sample”, “case” or “test set” are used interchangeably and mean the individual or case patient who is suspected of being or may be diseased and may ultimately be determined to be diseased or non-diseased.

As used herein, a “sample handling and processing marker,” “handling/processing marker,” “markers sensitive to variations in a sample handling and processing protocol,” “markers sensitive to pre-analytic variability,” and the like are used interchangeably to refer to a marker that has been found by methods described herein, to be sensitive to variations in a sample handling and processing protocol. “Sample handling and processing markers” may or may not include biomarkers.

Sample handling and processing markers can be identified from candidate markers in a control population of normal individuals. Samples obtained from said control population are analyzed for candidate markers to select candidate markers that are sensitive to variations in the sample handling and processing protocol. The variations include, but are not limited to, variations in sample processing time, processing temperature, storage time, storage temperature, storage vessel composition, and other storage conditions, prior to sample assay; variations in the method used to extract the sample from the normal individual, including, but not limited to exposure of the sample to oxygen, bore size of needle used for venipuncture, collection device, collection tube additives; variations in sample processing that include, but are not limited to, centrifugation speed, temperature and time, filtration and filter pore size; collection receptacle or vessel, method of freezing; and the like. Those candidate markers that are identified as substantially sensitive to variations qualify as sample handling and processing markers. The candidate markers comprise a variety of molecules including small molecules, peptides, proteins and nucleic acids.

In some cases, it can be desirable to distinguish in the selected handling/processing markers to remove those that can also be a disease marker or a marker for a particular disease at issue in the assay. On the other hand, it may not be necessary to eliminate a handling/processing marker in such circumstances, if the number of handling/processing markers to be used is larger, e.g., greater than any of about 20, 30, 50 or more.

As used herein, “determining”, “determination”, “detecting” or the like used interchangeably herein, refer to the detecting or quantitation (measurement) of a molecule using any suitable method, including fluorescence, chemiluminescence, radioactive labeling, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like. “Detecting” and its variations refer to the identification or observation of the presence of a molecule in a biological sample, and/or to the measurement of the molecule's value.

As used herein, a “biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, serum and dried blood spots collected on filter paper), sputum, tears, mucus, nasal washes, nasal aspirate, breath, urine, semen, saliva, cyst fluid, meningeal fluid, amniotic fluid, glandular fluid, lymph fluid, nipple aspirate, bronchial aspirate, pleural fluid, peritoneal fluid, synovial fluid, joint aspirate, ascite, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), lavage, fluid aspiration and a fine needle aspirate biopsy procedure. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, it should be realized that a biological sample can be derived by taking biological samples from a number of individuals and pooling them or pooling an aliquot of each individual's biological sample.

“Cell Abuse” includes, but not limited to, cellular contamination, cellular lysis, cellular fragmentation, cell fragments, internal cellular components and the like.

“Rejecting a sample” as used herein, can refer to a rejection of a subset, group or collection to which the sample belongs.

As used herein, a “SOMAmer” or “Slow Off-Rate Modified Aptamer” refers to an aptamer having improved off-rate characteristics. SOMAmers can be generated using the improved SELEX methods described in U.S. Publication No. 2009/0004667, now U.S. Pat. No. 7,947,447, entitled “Method for Generating Aptamers with Improved Off-Rates.”

In the subject application, the measurements of marker proteins for sample handling and processing have been measured and found to have definite and reproducible behavior with respect to variations in sample collection and preparation.

A central idea here is to use some of the many processing and handling marker proteins which can be measured in each sample, to provide graded responses to variations in the sample collection and steps of sample preparation. In this sense, these handling/processing marker protein signals can be used, for example, to monitor past events in blood sample processing such as delay before centrifugation and delay before decantation. This is different from monitoring the degradation of the biomarker proteins of interest directly, and can be both more sensitive and informative over a wide range. By using the methods described herein, the likely quality of a sample in regard to the changes post draw in specific biomarker proteins of interest can be characterized by applying the handling/processing markers' known sensitivities for each process variation, to the estimated values of the biomarkers. Monitoring of sample processing and handling markers can also be used to correct for the estimated effects of each variation in disease biomarkers by subtracting the sample handling component from the apparent protein concentration. These sample handling and processing biomarker measurements can be used to characterize samples prior to assessment of biomarkers of disease by a variety of measurement systems, including antibody assays, mass spectrometry, and the like.

In this way, some of the biological mechanisms of blood are used to act as clocks, timers and recording devices. For this technique to work, we must be able to distinguish between in vivo biological activation of the various mechanisms, and the activation which occurs after the blood has left the body, or “in vitro” changes. The main tool for distinguishing disease biomarker and handling/processing marker degradation in vivo from that incurred in vitro, is the ability to measure a great many proteins simultaneously, so that the sample can be characterized not merely for a single sample handling/processing variation, but for several. Correlated protein measurements indicative of particular sample handling protocol variations provide a panel of sample handling/processing markers.

The metrics delivered on each sample by our system enables one to reject sets of samples from clinical sites by evaluating a few samples to discover that the sample handling and processing techniques at one or more sites or in some fraction of the samples would have made it hard to measure differences in biomarker proteins of interest. That is, the metrics permit the determination of whether the samples at issue will conceal the true biology of health or disease due to sample handling effects, or whether the sample handling effects would produce a “false positive” biomarker result that was not really a reflection of the underlying biology of health or disease. The sample collection/processing metrics have also provided a window into reliable and robust biomarker discovery. By selecting groups of samples with consistent sample preparation metrics, unintended bias can be minimized and disease specific biomarker discovery enhanced. The metrics can also be used to correct mild sample handling effects by comparison to well collected standard samples. In clinical use, the sample handling metrics can be used to advise sites on their collection procedures, in order to reject some samples before expensive further evaluation, and in order to adjust the measurements or report provided to reflect any uncertainty due to sample handling.

In short, it is now possible to:

1. Determine the form and quantify extent of sample handling variation between samples. This permits the sample set to be triaged and separate out the samples suitable for biomarker discovery.

2. Identify or establish preferred sample handling/processing protocol to substantially reduce or minimize variation among samples.

3. Similarly, the sample handling/processing values of collection sites or batches of samples can be compared to reference sample handling/processing biomarker values to determine if individual sites are compliant with the preferred collection protocols.

4. Sample sets can be examined and compared to reference sample handling/processing biomarker values to determine the extent of expected handling and processing variation which may exist between case and control samples. In this way, subsets of samples can be chosen for comparison on the basis of similar sample collection conditions so that the biomarkers that are identified are a reliable reflection of the underlying biology.

5. Individual samples can be rejected for a diagnostic test if it is determined that the sample was not collected in manner that complies with a preferred handling/processing protocol.

6. The protein measurements of one or more case samples can be adjusted to reflect the sample handling/processing variability.

7. A robust subset of proteins which are less sensitive to sample handling/processing variability can be chosen for clinical or commercial use.

Thus, the invention comprises a method for quantifying the effect of deviations from ideal blood sample collection conditions. This method comprises the identification of biological processes which are influenced by variation in the steps involved in blood sample draw and handling, prior to proteomic assay measurement. These biological processes are monitored by specific lists of analyte (e.g., protein) measurements which are uniquely identified with such processes and which can be monitored. These protein lists are applied quantitatively using projections of logarithmic measurements of protein abundance using protein coefficients specific to each protein being measured. The scores from these projections known as Sample Processing marker SMVs (sample marker variation) can be used to assess the procedural variation blood sample collection on a per sample and per group of samples basis.

In one aspect, the subject invention protects the method by which SMV coefficients are created. Specifically, a method has been identified for quantifying the effect of deviations from ideal blood sample collection conditions. This method comprises the identification of biological processes which are influenced by variation in the steps involved in blood sample draw and handling, prior to proteomic assay measurement. These biological processes are monitored by specific lists of protein measurements which are uniquely identified with such processes and can be monitored by us. These protein lists are applied quantitatively using projections of logarithmic protein of measurements of protein abundance using protein coefficient specific to each protein being measured. The scores from these projections known as SMVs can be used to assess the procedural variation blood sample collection on a per sample and per group of samples basis. These biological processes can be used to monitor variations in blood sample collection conditions and the specific protein vectors can be used to monitor and quantify such biological processes. This provides a quantification of the sample collection variation which is recorded in the sample itself and does not need independent monitoring of variables such as times, temperatures, centrifugation speed; at the time of collection.

To identify the SMV protein components, targeted experiments were used that involved biochemical manipulation of specific biological processes, such as complement activation, platelet activation and cell lysis. These experiments are combined with experiments which alter the conditions the blood sample collection in a manner consistent with clinical practice to uniquely identify biological processes which may be used to quantitatively assess the variation in a clinical sample collection on a per sample basis.

The techniques described herein can be used to evaluate the samples as to the quality of the measurements of proteins involved directly in these biological processes. This provides quantitative measurements of sample quality which can be applied to inform decisions concerning measurements of proteins in these samples that can be affected by sample handling variation but are not simply linked directly to the biological processes that are measured here. For example, general proteolytic activity may be affected by activation of complement and lysis of cells. However, the affected proteins do not form a simple closed group or process and cannot be used to monitor complement and cell lysis since other proteins may have many reasons to vary between samples that are unconnected with sample handling variation, such as disease processes or renal function.

The use of a set of proteins with coefficients to monitor the biological processes and indirectly the variation in sample collection conditions, is an invention which has an advantage over a single protein in that it is less likely to suffer from individual variation and forms an ensemble of measurements which can be interpreted to give a robust estimate of the biological process activation. The use of log scaled measurements permits the monitoring of the relative fold change in the biological process activation and can be simply compared to reference samples using a difference corresponding to a ratio in linear space. This use of logarithms also implicitly scales the proteins measurements such that the differing ranges of concentrations between proteins in the set or vector are automatically normalized when using a reference sample.

The direct application of the SMV calculations to an individual blood sample provides scores which may be interpreted in terms of the biological process or indirectly the deviation of the specific sample collection conditions from the ideal conditions of the reference sample. These scores can then be used to define which samples meet criteria or fall within acceptable limits. This information can be used to reject individual samples. Rejecting individual samples is important during biomarker discovery in order to avoid assigning variation in protein abundance to the disease or process which is under investigation for biomarker discovery when such variation may have been caused by some set of individual set of samples being treated under a different sample collection protocol or conditions.

The SMV scores for individual samples may be used to group sets of samples that correspond to specific ranges of sample collection parameters. This allows one to define matched sets of samples where samples from one set have comparable sample collection procedures and parameters to samples from a previous or different collection study. This ability to form matched sets is invaluable in comparing between groups of samples that may have been collected under different conditions. The SMV scores calculated from individual samples may also be used to correct for variation in the sample handling if the correlated variation in other proteins can be determined and a mathematical model built upon the variation in each protein affected by the processes leading to the variation between samples with different SMV scores.

The rejection of individual samples on the basis of their SMV scores allows the performance of more sensitive biomarker discovery since we know that the differences between samples collected from clinically different individuals refer to the differences between those individuals, not between differences in how the samples were collected. Diagnostic tests involving proteins abundance may be misleading if that variation is due to procedure by which the blood sample was collected and not due to the clinical state of the individual. This is avoided by rejecting samples which do not meet SMV score thresholds corresponding to reasonable sample collection procedural variation.

Many existing sample collections are systematically damaged by variations in sample collection procedure. The SMV scores may be used to quantify such variation within a sample collection or between sample collection sites and can be used to reject whole studies on the basis of variation which may mislead the investigator, such as systematic variation in sample collection between case and control. It is necessary that only a subset of the collection be measured to assess such variation; large savings are possible, in the case that a sample collection is deemed unacceptable. It also possible to monitor sample collection during the sample acquisition stage of a study and thus provide corrective advice and detect non-compliance with study protocols. To monitor variation in existing or ongoing studies it is only necessary to measure some sub-sample of the entire collection.

These techniques for monitoring and assessing sample collection variation may be applied to the optimization of study protocols and may be applied to the economic maximization of large sample collection efforts such as bio-banks where the cost of employing special sample collection equipment and vessels may be compared with an accurate assessment of the variation and damage due to operating with a less expensive protocol.

In some cases, it not possible to obtain pristine sample collections, possibly due to the retrospective nature of most common collections of biological samples. And some comparisons may perforce occur between samples collected at different sites and between groups of samples collected at different times. These sample collections will show differences in collection procedure which will cause variations in the proteomic profiles which will be confounded with the intended differential clinical comparison. By creating matched sets between the sample groups, it is possible to compare equivalently collected subsets of samples.

The measurement of protein analytes in plasma samples can be significantly affected by the protocol used to collect and handle the sample. Deviations from a specified sample collection and/or handling protocol can lead to changes in protein levels within the sample or other systematic effects on measurements that result in changes to signals for many analytes, including negative controls. Such deviations may occur irrespective of the type of assay used to measure the protein analytes.

In order to assess the quality of a set of clinical samples, the effects for the most obvious deviations from protocol have been characterized. Variability in protein composition as a function of time has been assessed between sample collection and spinning. Further, variability in protein composition as a function of time has been assessed between sample spinning and the time to decanting of the sample.

Signatures for sample mishandling have been identified that can be used as a quantitative classifier for assessing collections of clinical samples. Further, metrics have been produced for each analyte that capture the sensitivity of that analyte's measurements to deviations from collection protocol, particularly with respect to delay between sample collection and spinning and delays between sample spinning and sample decanting.

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1 Sample Collection

Plasma samples were collected from a group of eighteen individuals in which all sample collection variables were held constant at the defined protocol with the exception of the variable of interest. Multiple tubes were drawn from the same set of individuals to assess the variation in responses among different individuals.

### Sample Collection—Steps

1. The expiration date on a lavender top EDTA tube was checked. If expired, it was replaced with a new one.
2. The lavender top EDTA tube was labeled with the correct participant ID and collection date.
3. Venipuncture was performed per institutional guidelines, in compliance with standard health and safety procedures.
4. The lavender top EDTA tube was filled completely with the sample from the venipuncture.
5. If labs were being drawn at the same time, the tubes were collected in the following order:
   a. Serum
   b. Citrate Plasma
   c. Heparin Plasma
   d. Lavender top EDTA Plasma
6. The lavender top EDTA tube was inverted 8-10 times and placed upright in a rack.

### Time-to-Spin

Samples were collected in vacutainer tubes and inverted as described in the Sample Collection—Steps above. Subsequently, six different times were allowed to elapse before samples were spun for each of the eighteen individuals, namely, 0, 0.5, 1.5, 3, 9, and 24 hours. Lavender top EDTA tubes were spun at 2200×g (Not RPM) for 15 minutes. A Microfuge tube was labelled with the correct participant ID. 1.0 ml of plasma was pipetted into a Microfuge tube. Only the plasma layer was drawn off. Care was taken to not disturb the buffy coat when aliquoting, by leaving some plasma behind and avoiding the cell layer. The top on the Microfuge tube was closed and placed in a −80° C. freezer.

### Time-to-Decant/Freeze

Samples were collected as described in the Sample Collection—Steps and Time-to-Spin above through sample spinning. Subsequently, six different times were allowed to elapse before the spun samples were decanted and frozen for each of the eighteen individuals, namely, 0, 0.5, 1.5, 3, 9, and 24 hours.

### Univariate Analysis

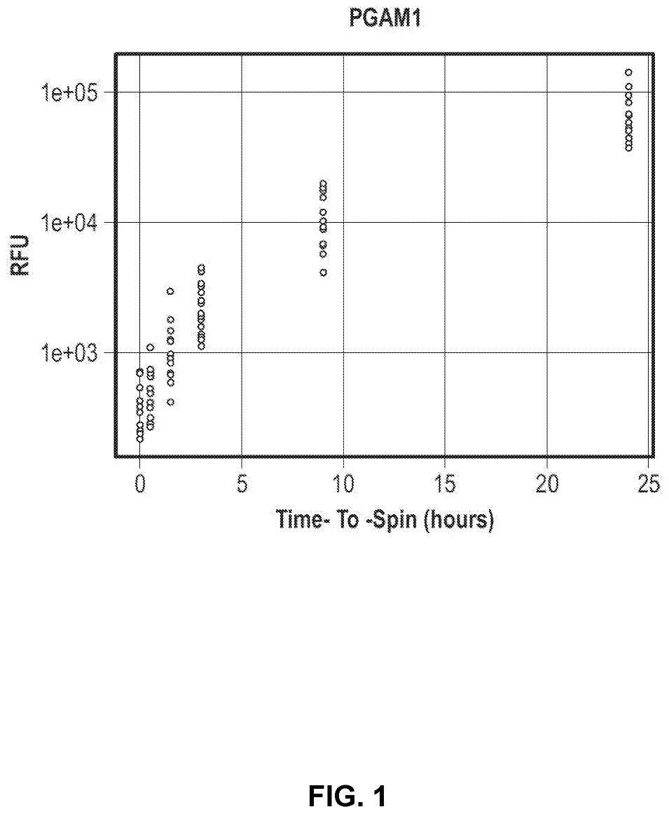

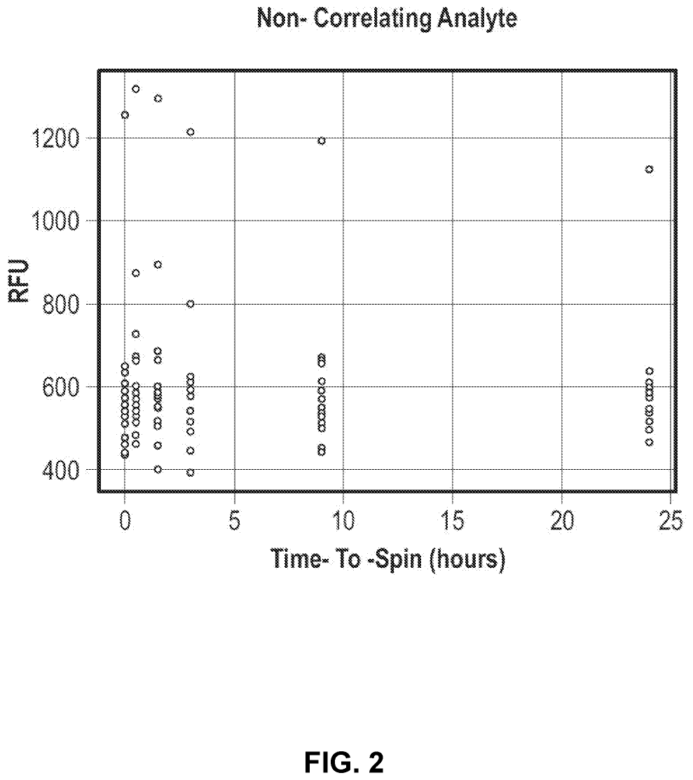

Prior to model generation univariate analysis on all analyte signals with respect to time-to-spin/time-to-decant was done to reduce the number of features (analytes) used in model building (FIGS. 1-2).

Pearson correlation (Equation 1) of the 18 individual's RFU was calculated for each of the ˜5K analytes to access the general functional affect with varying time-to-spin/time-to-decant.

$$• = \frac{• • • ( • , • )}{{•}_{•} {•}_{•}} \qquad ( 1 )$$

Although there is a continuum of behavior analytes can be characterized as high discriminatory properties (FIG. 1) with Pearson correlation coefficient>0.95 or with very low discriminatory properties with correlation coefficients<1E-3 (FIG. 2) showing virtually no change with time-to-spin in the 18 individuals.

Summary statistics of a few of the analytes with high time-to-spin/time-to-decant correlation are displayed in Table 1 & 2. Table 3 ranks time-to-spin/time-to-decant analyte importance. There are qualitative groups of analytes in the time-to-spin model; those showing negative or positive correlative shifts in RFU with increasing time-to-spin and those with varying degrees of time-to-spin response. Table 4 displays correlation between the level of the analyte measured and the time-to-spin (e.g., the measured levels of SHH decrease as the time from collection to spin increases (negative correlation)).

Using the calculated analyte correlation, we reduced the number of potential markers that could be useful in time-to-spin/time-to-decant classification from ˜5K to ˜100 which have calculated Pearson correlation>10.71. Using this initial set of analytes we perform further feature reduction when constructing the classifier.

### Example 2 Classifier Generation

Random forest classifiers were chosen to generate sample handling models. A brief introduction to random forests, its implementation using SOMAscan data, and its strength over another machine learning technique follows.

### Random Forest Model

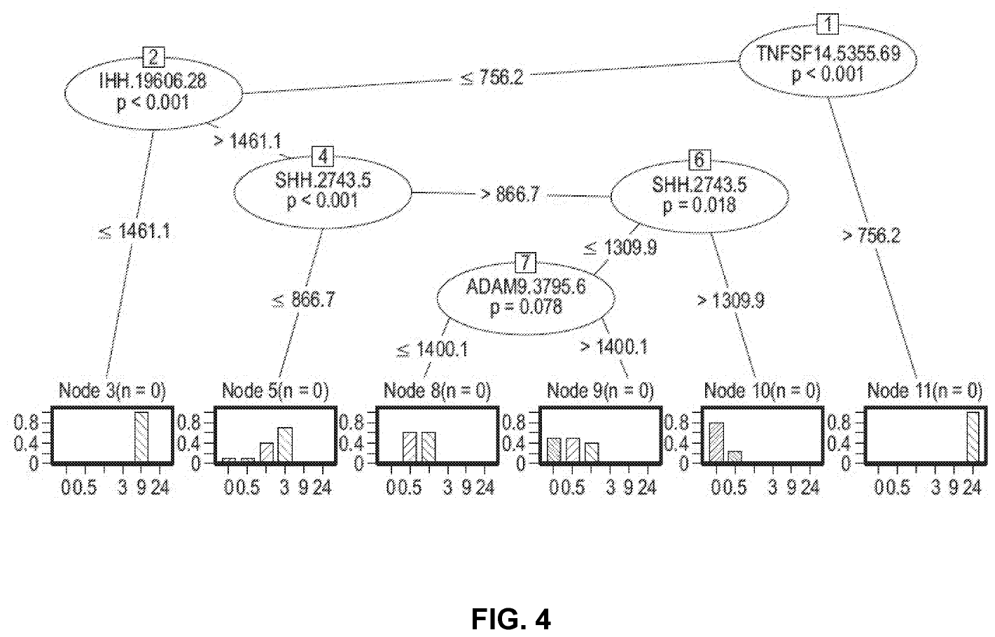

Briefly, a random forest is a collection of many (hundreds) decision trees as in the example below (FIG. 4). RFU levels at a node will split a tree in two directions —either leading to an endpoint and classification prediction or to another node where an additional analyte RFU value will split the tree again and lead further down multiple branches.

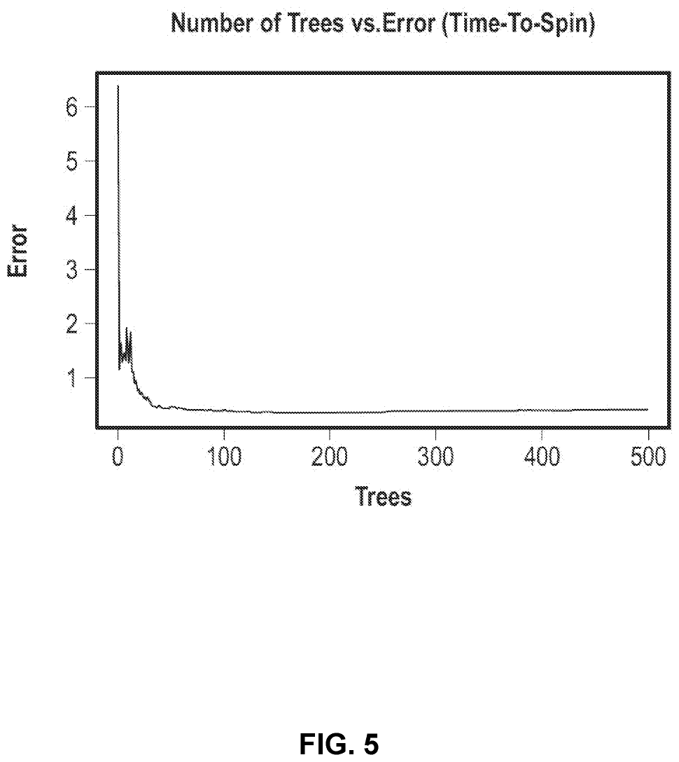

A benefit of a random forest is where one decision tree will be prone to prediction errors, such as multiple incorrect binning in FIG. 4, the average prediction on hundreds of trees will reduce the error on any given prediction (FIG. 5).

### Model Generation

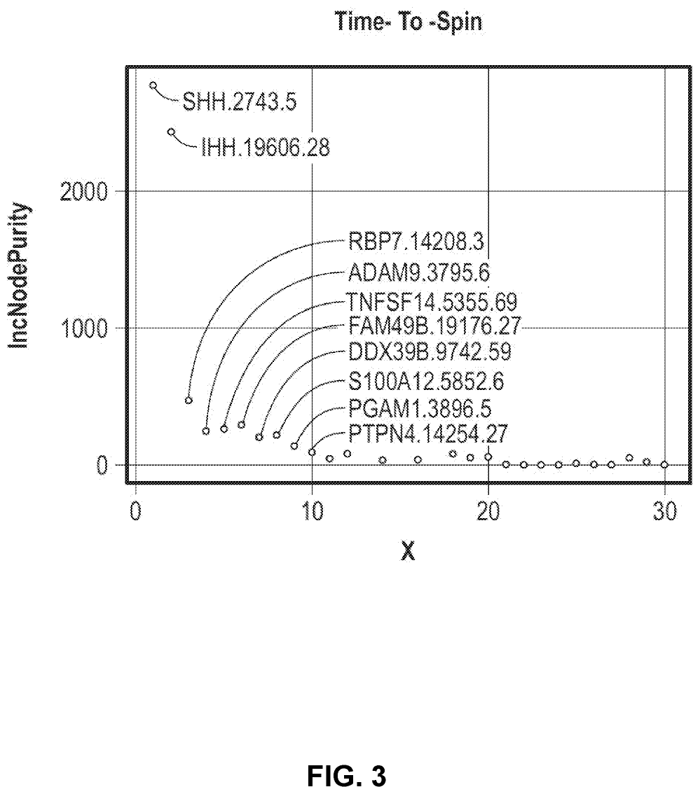

The random forest model was trained using Caret (Kuhn, M. (2008). Caret package. Journal of Statistical Software, 28(5)) and random Forest (A. Liaw and M. Wiener (2002). Classification and Regression by randomForest. R News 2(3), 18-22) package in R on log₁₀ transformed RFU data. We performed further feature reduction by evaluating the IncNodePurity (Gini Index in classification), a measure of the relative importance of each analyte on the performance of the model. From here we further reduced the features to generate a model which includes 10 of the most important analytes in time-to-spin/time-to-decant (Table 3, FIG. 3).

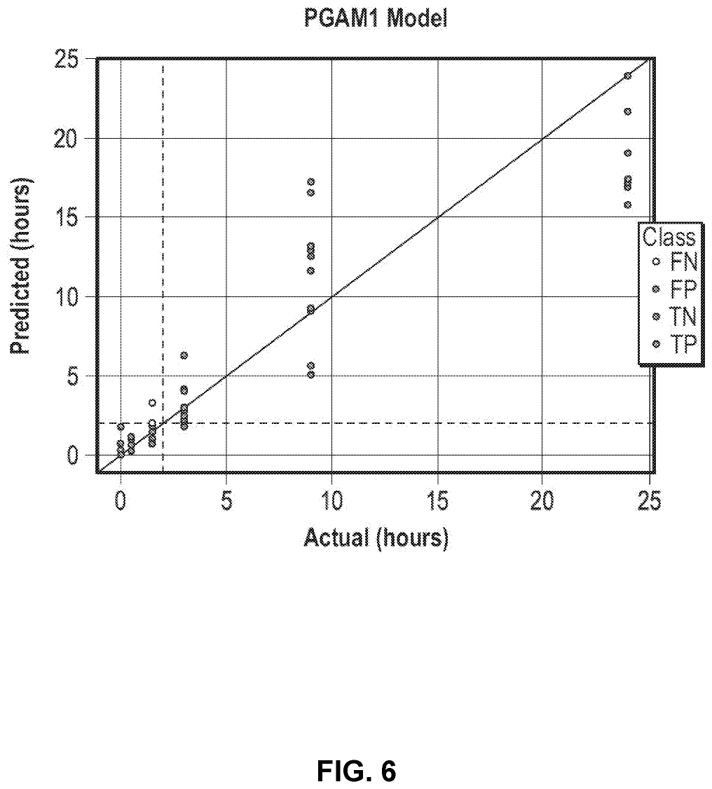

When evaluating model performance using individual analytes (FIG. 6; PGAM1 model) we use two metrics. First by assessing the prediction time against the true time using root mean square error (RMSE) or by thresholding sample times by what we deem as a well collected sample (true time-to-spin/time-do-decant less than 2 hours) or a poorly collected sample (true time greater than 2 hours). Using this binary classification, we can assign predictions as true positive (TP), meaning the prediction time accurately describes a well collected sample, true negatives (TN), meaning the prediction time accurately describes a poorly collected sample, and the cross terms false positive (FP) and false negative (FN). Using only PGAM1 as a predictor for time-to-spin (Figure X) we observe good levels of sensitivity/specificity in the binary classification system, although at longer time-to-spin we often underestimate or overestimate the true value.

### Model Stability

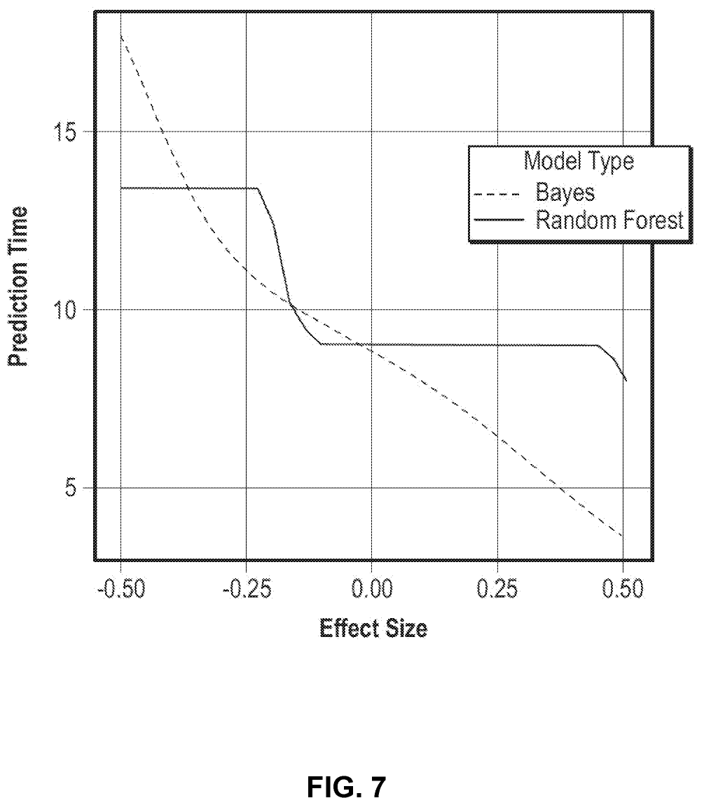

An additional benefit the random forest model shows is stability in the event of assay noise. Consider a sample with true time-to-spin of 9 hours (FIG. 7). If the RFU value on a single analyte (SHH in this example) is increased/decreased by some effect size the model prediction is more stable (solid line curve) than a similar Naive Bayes model (dashed line curve).

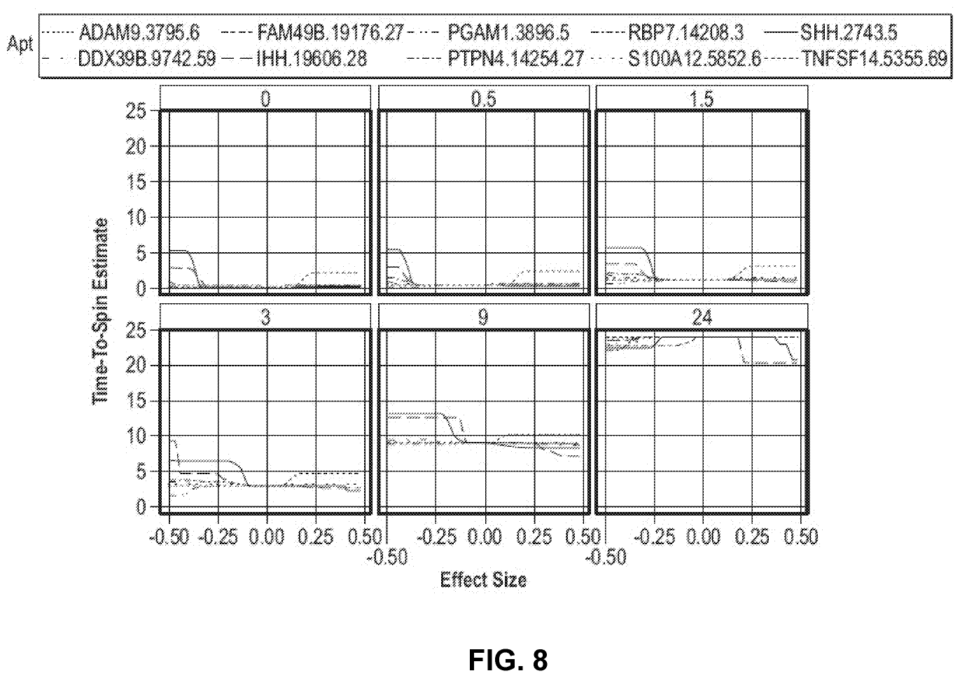

When adjusting the RFU on each of the 10 analytes independently for a given sample and time-to-spin we observe there is a relative stable point around which the actual prediction does not vary significantly (FIG. 8). At more extreme changes to analyte signals we observe modest jumps to prediction times and not continuous changes.

### Binary Classification Performance

Using a pre-defined cutoff time of 2 hours—with samples having actual time-to-spin/time-to-decant of below 2 hours as “good” samples and samples with greater than 2 hours as “compromised/bad” samples overall sensitivity and specificity was defined to each model predictions against what is known to be the actual class. Using this binary classification, predictions were assigned as true positive (TP), meaning the prediction time accurately describes a well collected sample, true negatives (TN), meaning the prediction time accurately describes a poorly collected sample, and the cross terms false positive (FP) incorrectly describing a poorly collected sample and false negative (FN) incorrectly describing a well collected sample. For example, based on the PGAM1 model (FIG. 6) at actual time of 1.5 or 3 hours Table 5 Binary Classification Performance was produced.

The confusion matrix contained the following information:

Reference

| Prediction | False | True |
|---|---|---|
| False | 15 | 1 |
| True | 3 | 17 |

Where 17 samples were marked as true positive, 15 marked as true negative, 1 marked as false negative, 3 marked as false positive.

The sensitivity of a model is calculated as:

$$sensitivity = \frac{True Positive}{True Positive + False Negative}$$

$$sensitivity = \frac{True Positive}{True Positive + False Negative}$$

$$and the specificity calculated as \text{:}$$

$$specificity = \frac{True Negative}{True Negative + False Positive}$$

$$specificity = \frac{True Negative}{True Negative + False Positive}$$

For these 18 individuals at 2 timepoints the sensitivity/specificity correspond to:

$$sensitivity = \frac{17}{17 + 1} = 0.94$$

$$sensitivity = \frac{17}{17 + 1} = 0.94$$

$$specificity = \frac{15}{15 + 3} = 0.83$$

$$specificity = \frac{15}{15 + 3} = 0.83$$

The full sensitivity/specificity is calculated across the 18 individuals at the 6 time-to-spin/time-to-decant.

### RMSE Calculation

The root mean square error (RMSE) is a continuous measurement of performance calculated at the true time-to-spin against the predictions at each sample and time.

$$\sqrt{\frac{{\sum }_{i = 1}^{n} {( {Predicted}_{i} - {Actual}_{i} )}^{2}}{N}}$$

$$\sqrt{\frac{{\sum }_{i = 1}^{n} {( {Predicted}_{i} - {Actual}_{i} )}^{2}}{N}}$$

For the 18 individuals at an actual time-to-spin of 9 hours in the example PGAM1 marker, the numerator of this equation contains the data of Table 6.

Summing the square difference, and using N=18 samples, reduces the equation to:

$$\sqrt{\frac{219.09}{18}} = 3.488 hours$$

$$\sqrt{\frac{219.09}{18}} = 3.488 hours$$

Reducing the difference between the predicted time and actual time lowers the RMSE thus is a good indicator of model performance. An RMSE of 0 across all timepoints and samples would correspond to each prediction equal to the actual time-to-spin (i.e. a perfect predictor).

### Model Training and Performance

18 individuals were used in training models and evaluating model performance. “Good” samples were defined as having a predicted time-to-spin less than 2 hours to create a binary class system. In addition, the relative error associated with the predicted time against the actual time is an additional indicator of model performance (RMSE). Random forest models confine the predictions between 0 and 24 hours (where data is available). FIG. 6 demonstrates the performance of a model with a single analyte predictor, colored by whether a sample was correctly identified as a true positive, those with correctly predicted time-to-spin of less than 2 hours and the and the alternative class predictions, plotted against the true time-to-spin as indicator of the model accuracy.

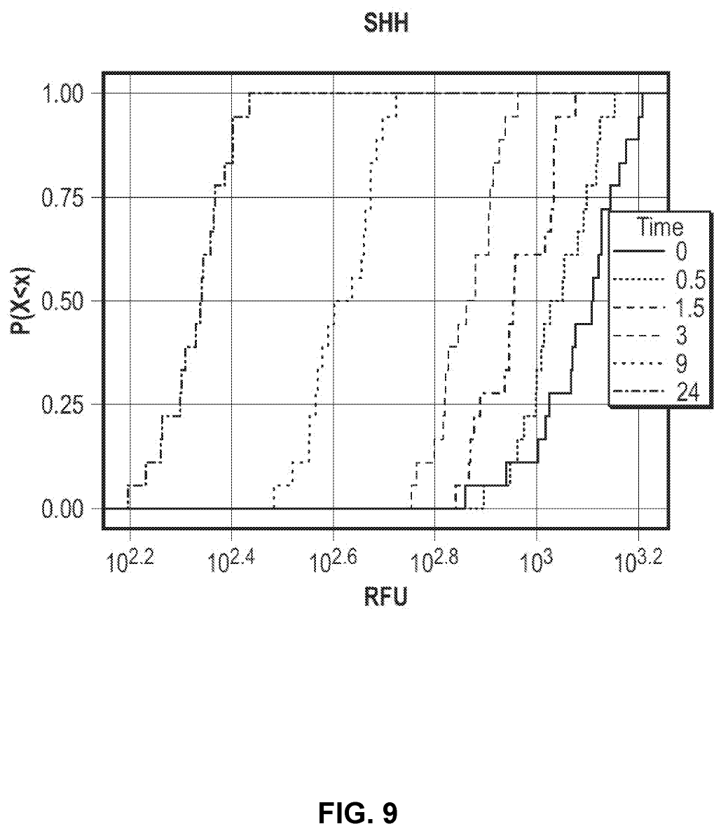

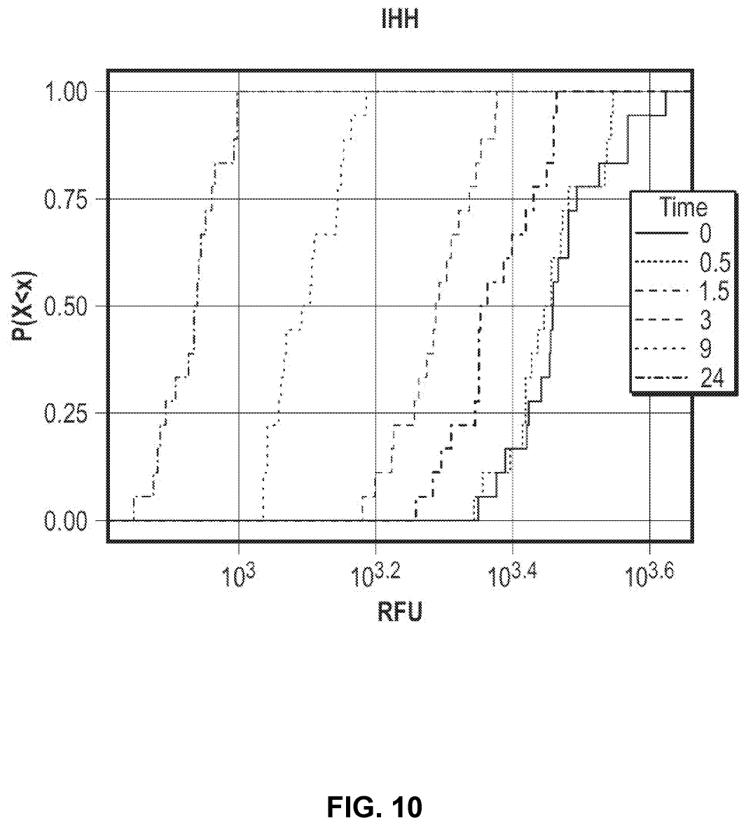

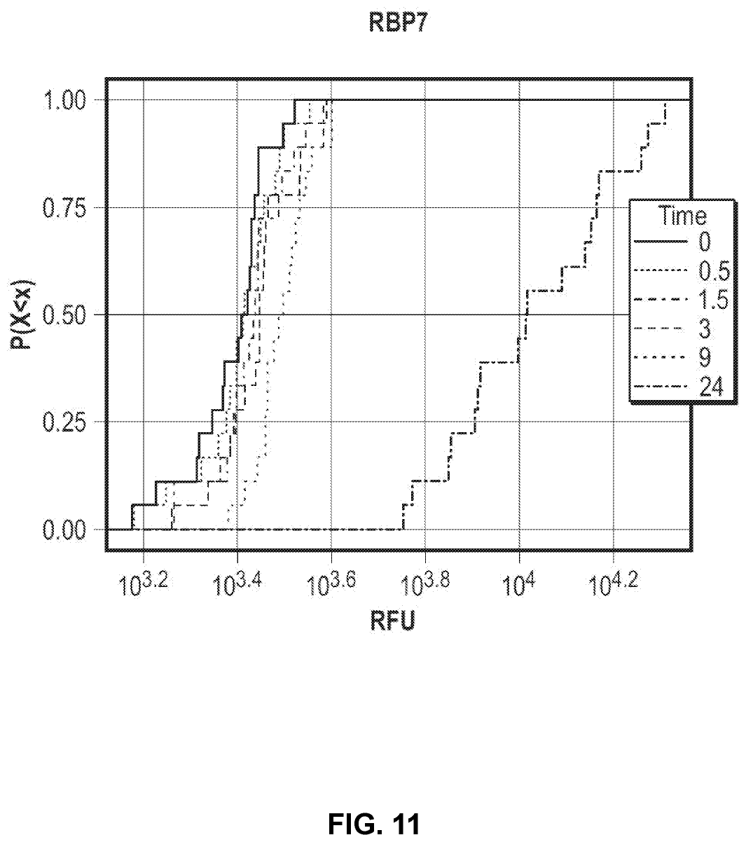

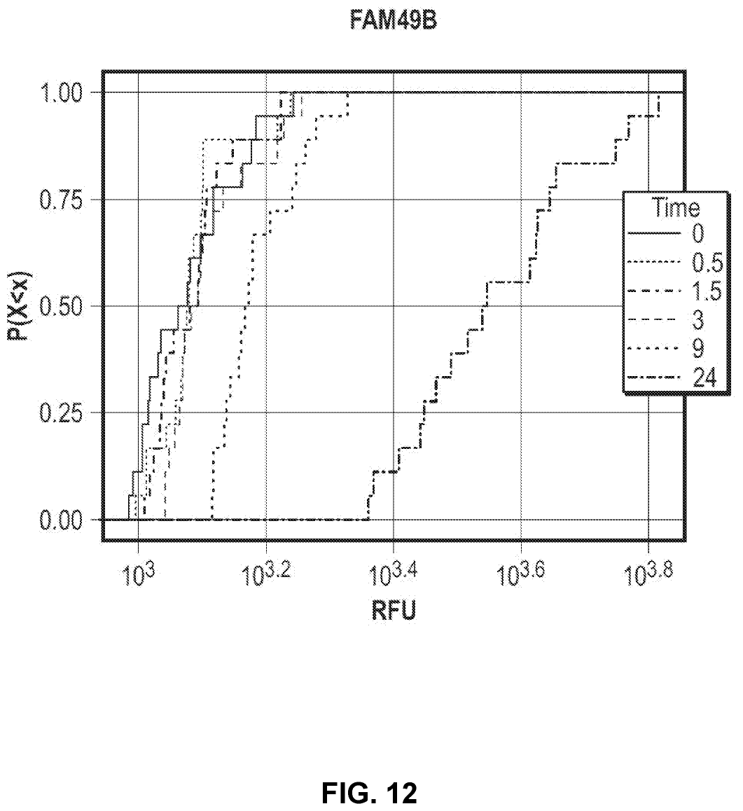

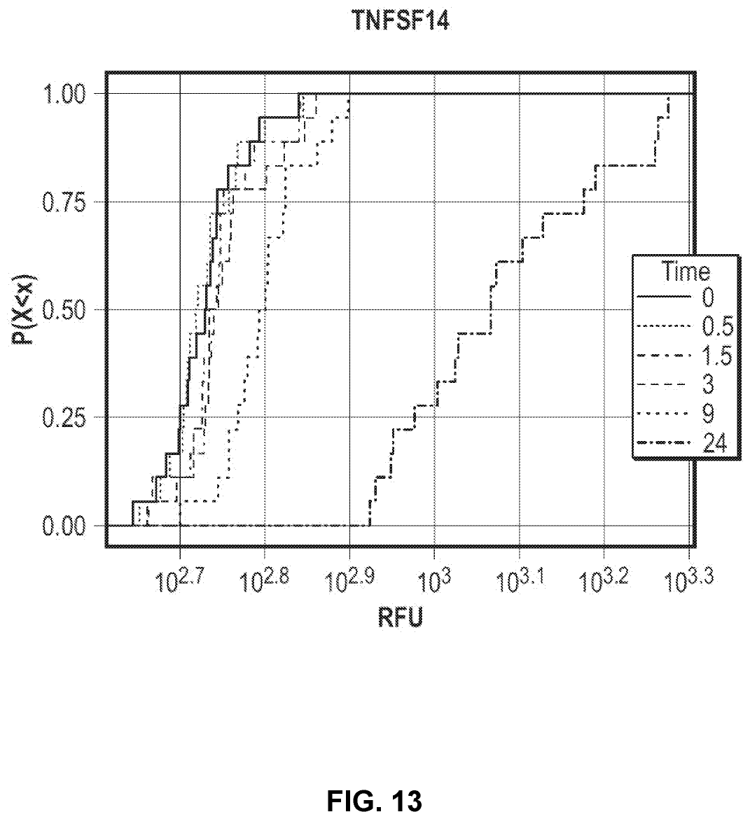

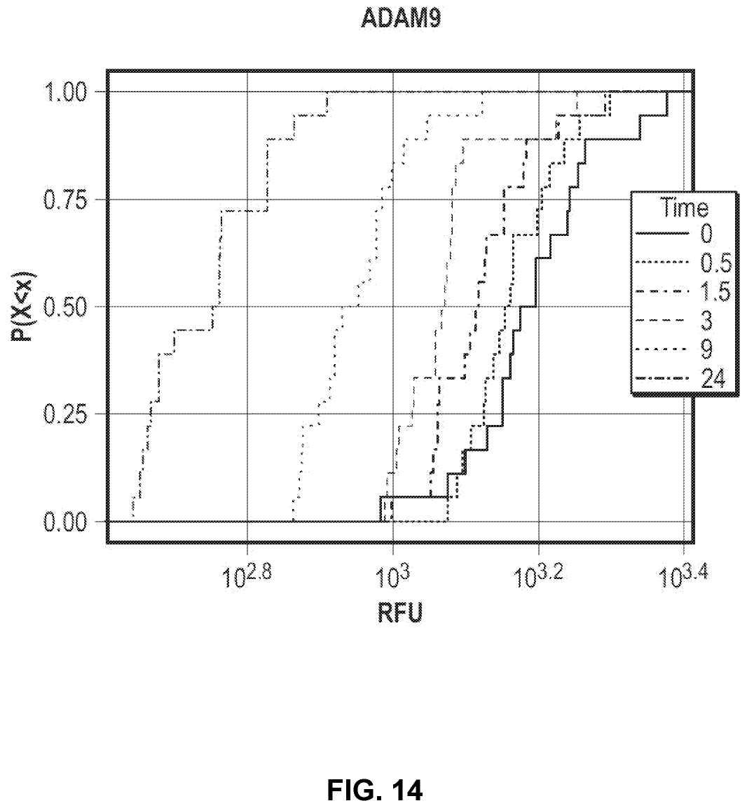

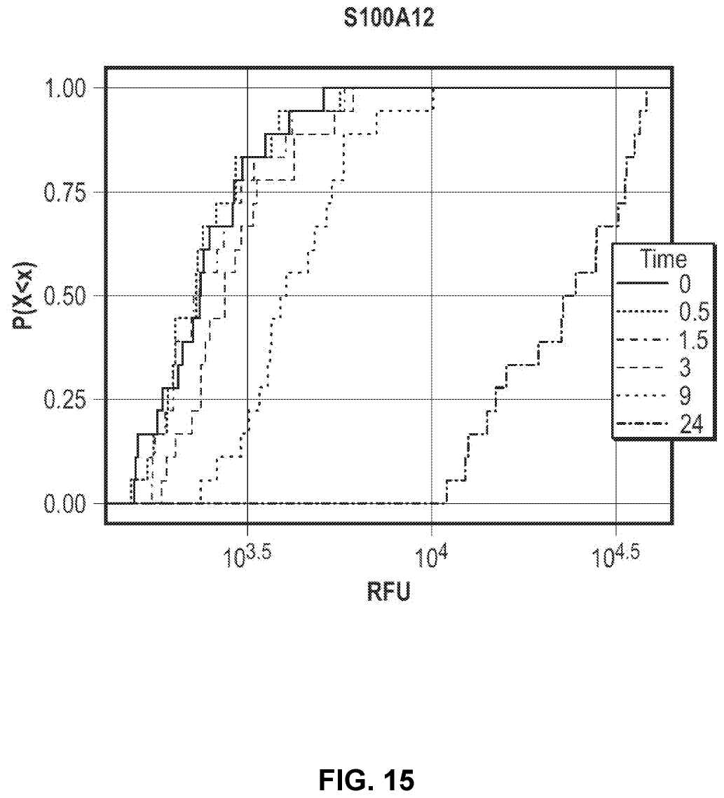

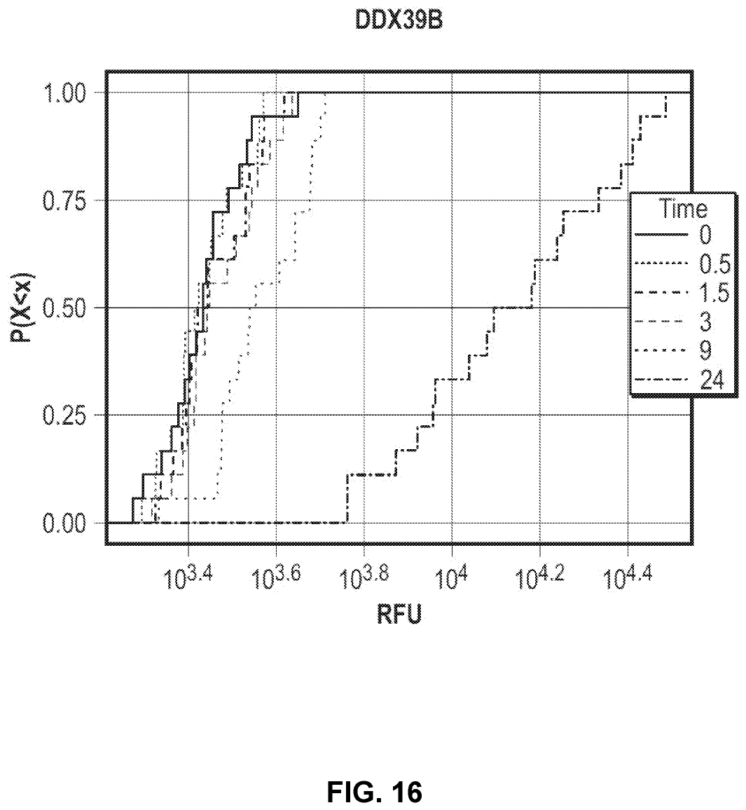

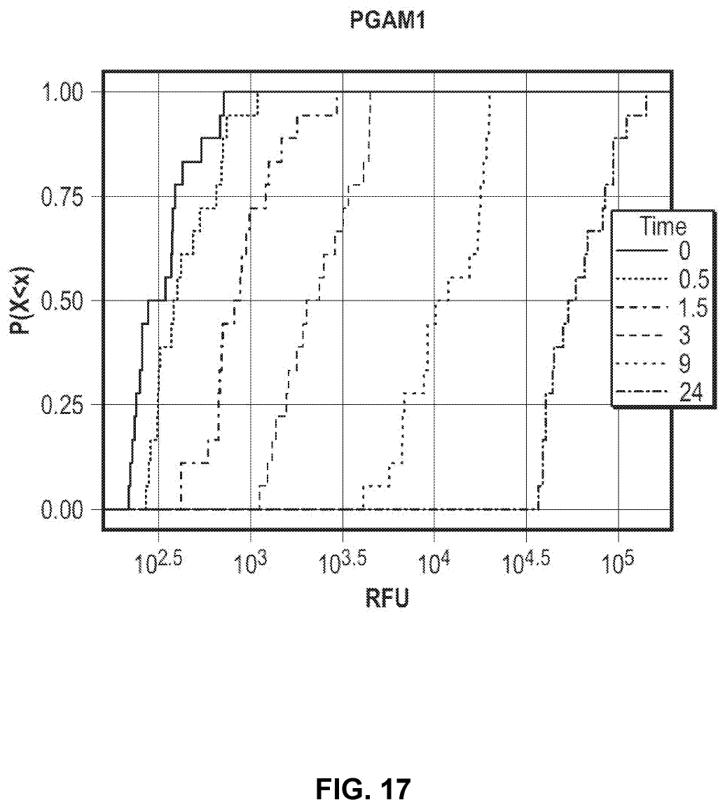

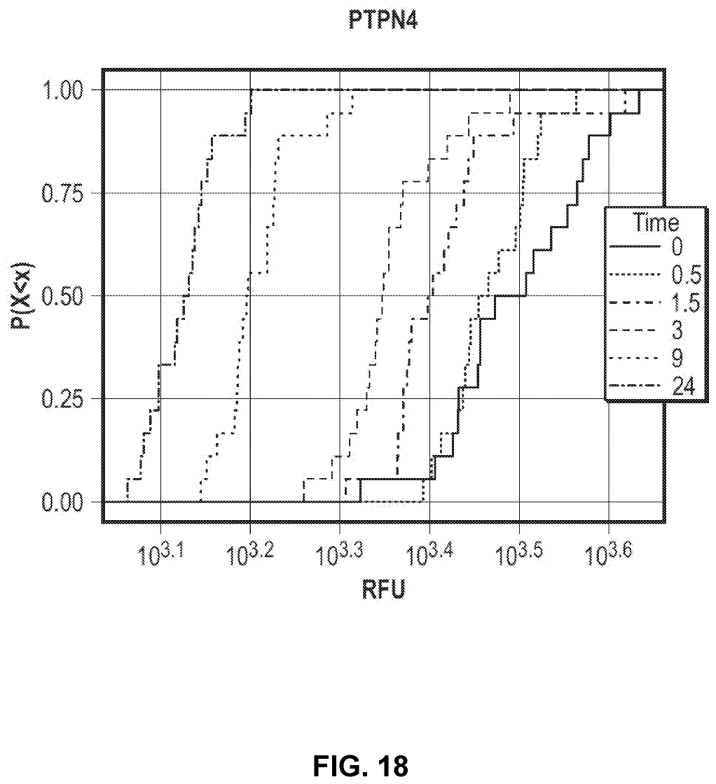

Cumulative distribution functions for 18 individuals with varying time-to-spin are found at FIGS. 9-18.

### Analyte Model Performance

Analysis to quantify performance of individual and combinations of analytes is summarized in Tables 7-24. Table 7 shows Time-to-Spin for Single Marker Model Performance for all predicted time points (0, 0.5, 1.5, 3, 9 and 24 hours sample sat prior to spinning). Table 8 shows Time-to-Spin for Two Marker Model Performance for all predicted time points (0, 0.5, 1.5, 3, 9 and 24 hours sample sat prior to spinning). Table 9 shows Time-to-Spin for Three Marker Model Performance for all predicted time points (0, 0.5, 1.5, 3, 9 and 24 hours sample sat prior to spinning). Table 10 shows Time-to-Spin Performance for Models with Sonic Hedgehog (SHH) for all predicted time points (0, 0.5, 1.5, 3, 9 and 24 hours sample sat prior to spinning). Table 11 shows Time-to-Spin Performance for Models with Indian Hedgehog (IHH) for all predicted time points (0, 0.5, 1.5, 3, 9 and 24 hours sample sat prior to spinning). Table 12 shows Time-to-Spin Performance for Models with ADAM9 for all predicted time points (0, 0.5, 1.5, 3, 9 and 24 hours sample sat prior to spinning). Table 13 shows Time-to-Spin Performance for Models with DDX39B for all predicted time points (0, 0.5, 1.5, 3, 9 and 24 hours sample sat prior to spinning). Table 14 shows Time-to-Spin Performance for Models with FAM49B for all predicted time points (0, 0.5, 1.5, 3, 9 and 24 hours sample sat prior to spinning). Table 15 shows Time-to-Spin Performance for Models with PGAM1 for all predicted time points (0, 0.5, 1.5, 3, 9 and 24 hours sample sat prior to spinning). Table 16 shows Time-to-Spin Performance for Models with PTPN4 for all predicted time points (0, 0.5, 1.5, 3, 9 and 24 hours sample sat prior to spinning). Table 17 shows Time-to-Spin Performance for Models with RBP7 for all predicted time points (0, 0.5, 1.5, 3, 9 and 24 hours sample sat prior to spinning). Table 18 shows Time-to-Spin Performance for Models with S100A12 for all predicted time points (0, 0.5, 1.5, 3, 9 and 24 hours sample sat prior to spinning). Table 19 shows Time-to-Spin Performance for Models with TNFSF14 for all predicted time points (0, 0.5, 1.5, 3, 9 and 24 hours sample sat prior to spinning). Table 20 shows Time-to-Decant for Single Marker Model Performance for all predicted time points (0, 0.5, 1.5, 3, 9 and 24 hours sample sat prior to spinning). Table 21 shows Time-to-Decant for Two Marker Model Performance for all predicted time points (0, 0.5, 1.5, 3, 9 and 24 hours sample sat prior to spinning). Table 22 shows Time-to-Decant for Three Marker Model Performance for all predicted time points (0, 0.5, 1.5, 3, 9 and 24 hours sample sat prior to spinning). Table 23 shows Time-To-Spin Performance for Models with the combination of IHH, RBP7, ADAM9 and PTPN4 for all predicted time points (0, 0.5, 1.5, 3, 9 and 24 hours sample sat prior to spinning). Table 24 shows Time-To-Spin Performance for Models with analyte combinations, some of which comprise PGAM1 and/or PTPN4 and for all predicted time points (0, 0.5, 1.5, 3, 9 and 24 hours sample sat prior to spinning).

### Analyte Model Clustering

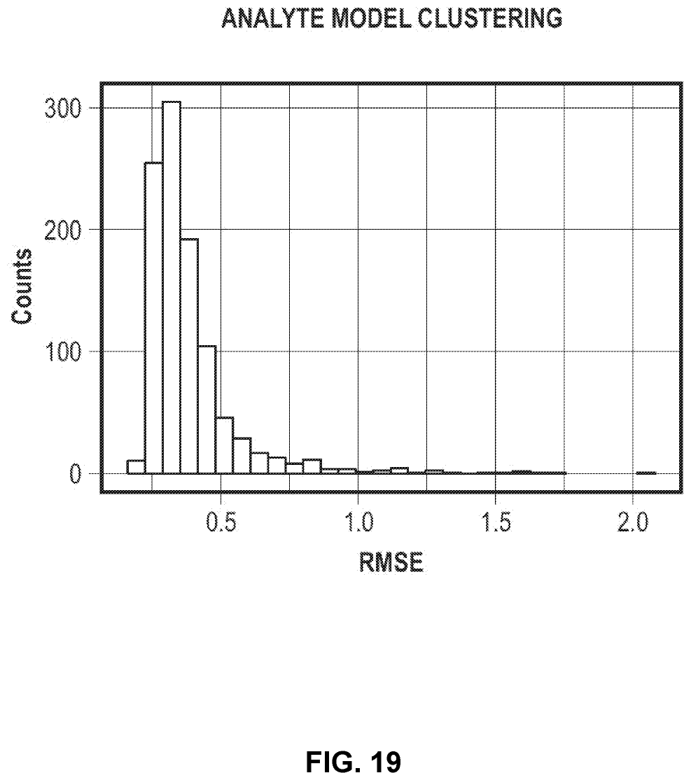

The performance of each model was quantified using the RMSE for the predicted time-to-spin against the true time-to-spin for each individual and timepoint. FIG. 19 shows the distribution of RMSE values for the 1023 models. The distribution is split into model performance of 4 groups—between 0 and 0.35 RMSE for high performing models, 0.35 to 0.5 for mid range performance, 0.5 to 1 for low performance and 1 to 2 for very low performing models.

### Analytes Used

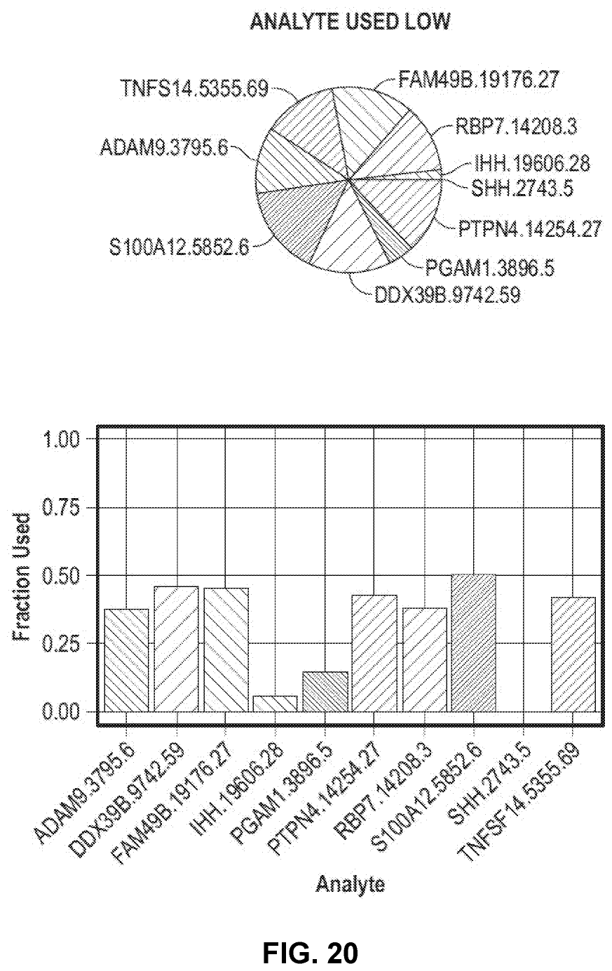

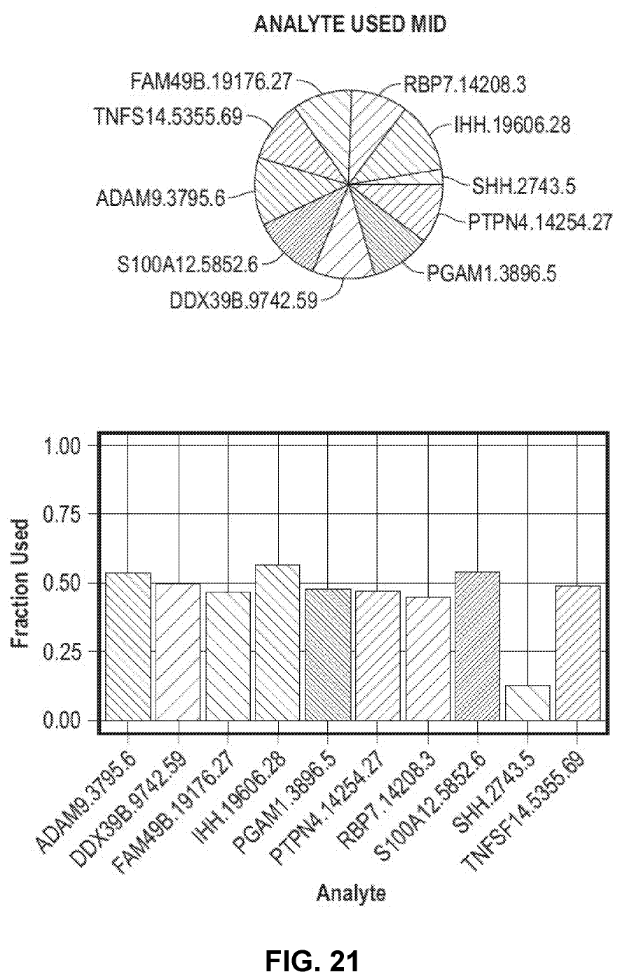

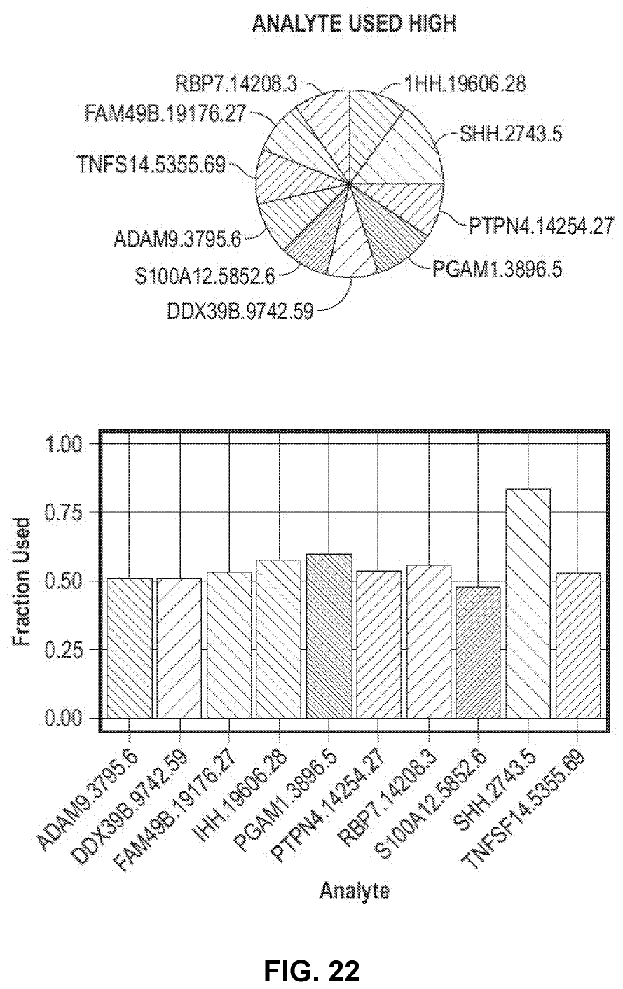

The fraction of times an analyte was used in each grouping of model performance was quantified to elucidate the importance of each analyte on model performance, as illustrated at FIGS. 20-22.

### Analyte Groups in Model Performance

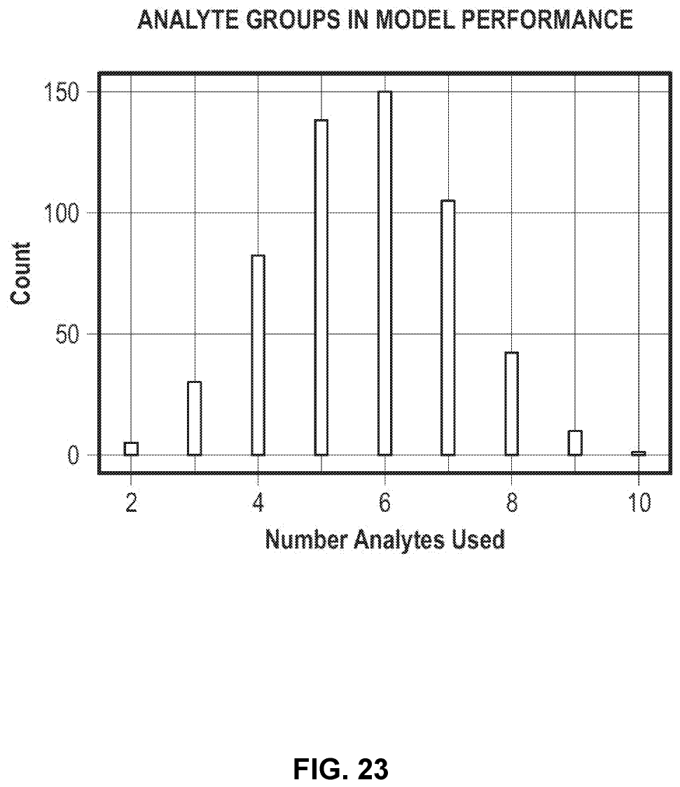

Of the high performing models the distribution of the number of analytes required is shown at FIG. 23. A good performing model can use as little as 2 analytes to all the analytes.

### Example 3. Multiplexed Aptamer Analysis of Samples

This example describes the multiplex aptamer assay used to analyze the samples and controls for the identification of the sample collection/processing variability markers set forth in Table 1.

### Multiplex Aptamer Assay Method

All steps of the multiplex aptamer assay were performed at room temperature unless otherwise indicated.

### Preparation of Aptamer Master Mix Solutions.

5272 aptamers were grouped into three unique mixes, Dil1, Dil2 and Dil3 and corresponding to the plasma or serum sample dilutions of 20%, 0.5% and 0.005%, respectively. The assignment of an aptamer to a mix was empirically determined by assaying a dilution series of matching plasma and serum samples with each aptamer and identifying the sample dilution that gave the largest linear range of signal. The segregation of aptamers and mixing with different dilutions of plasma or serum sample (20%, 0.5% or 0.005%) allow the assay to span a 10⁷-fold range of protein concentrations. The stock solutions for aptamer master mix were prepared in HE-Tween buffer (10 mM Hepes, pH 7.5, 1 mM EDTA, 0.05% Tween 20) at 4 nM each aptamer and stored frozen at −20° C. 4271 aptamers were mixed in Dil1 mix, 828 aptamers in Dil2 and 173 aptamers in Dil3 mix. Before use, stock solutions were diluted in HE-Tween buffer to a working concentration of 0.55 nM each aptamer and aliquoted into individual use aliquots. Before using aptamer master mixes for Catch-0 plate preparation, working solutions were heat-cooled to refold aptamers by incubating at 95° C. for 10 minutes and then at 25° C. for at least 30 minutes before use.

### Catch-0 Plate Preparation.

60 μL of Streptavidin Mag Sepharose 10% slurry (GE Healthcare, 28-9857) were dispensed into each well of the 96-well plates (Thermo Scientific, AB-0769). Beads were washed once with 175 μL of the Assay Buffer (40 mM HEPES, pH 7.5, 100 mM NaCl, 5 mM KCl, 5 mM MgCl₂, 1 mM EDTA, 0.05% Tween-20) and then 100 μL of the heat-cooled aptamer master mix was added to each well. Plates were incubated for 30 minutes at 25° C. with shaking at 850 rpm on ThermoMixer C shaker (Eppendorf). After 30 min incubation, 6 μL of the MB Block buffer (50 mM D-Biotin in 50 mM Tris-HCl, pH 8, 0.01% Tween) was added to each well of the plate and plates were further incubated for 2 min with shaking. Plates were then washed with 175 μL of the Assay Buffer, wash cycle of 1 min shaking on the ThermoMixer C at 850 rpm followed by separation on the magnet for 30 seconds. After wash solution was removed, beads were resuspended in 175 μL of Assay buffer and stored at −20° C. until use.

### Catch-2 Bead Preparation.

Before the start of the robotic processing of the assay, 10 mg/mL bead slurry of MyOne Streptavidin C1 beads (Dynabeads, part number 35002D, Thermo Scientific) used for Catch-2 step of the multiplex aptamer assay was washed in bulk once the MB Prep buffer (10 mM Tris-HCl, pH8, 1 mM EDTA, 0.4% SDS) for 5 min followed by two washes with Assay buffer. After the last wash, beads were resuspended at 10 mg/mL concentration and 75 μL of bead slurry was dispensed into each well of the Catch-2 plate. At the beginning of the assay, Catch-2 plate was placed in the aluminum adapter and placed in the appropriate position on the Fluent deck.

### Sample Thawing and Dilutions.

65 μL aliquots of 100% plasma or serum samples, stored in Matrix tubes at −80° C., were thawed by incubating at room temperature for ten minutes. To facilitate thawing, the tubes were placed on top of the fan unit which circulated the air through the Matrix tube rack. After thawing the samples were centrifuged at 1000×g for 1 min and placed on the Fluent robot deck for sample dilution. A 20% sample solution was prepared by transferring 35 μL of thawed sample into 96-well plates containing 140 μL of the appropriate sample diluent. Sample diluent for plasma was 50 mM Hepes, pH 7.5, 100 mM NaCl, 8 mM MgCl2, 5 mM KCl, 1.25 mM EGTA, 1.2 mM Benzamidine, 37.5 μM Z-Block and 1.2% Tween-20. Serum sample diluent contained 75 μM Z-block, the other components were the same concentration as in plasma sample diluent. Subsequent dilutions to make 0.5% and 0.005% diluted samples were made into Assay Buffer using serial dilutions on Fluent robot. To make 0.5% sample dilution, intermediate dilution of 20% sample to 4% was made by mixing 45 μL of 20% sample with 180 μL of Assay Buffer, then 0.5% sample was made by mixing 25 μL of 4% diluted sample with 175 μL of Assay Buffer. To make 0.005% sample, 0.05% intermediate dilution was made by mixing 20 μL of 0.5% sample with 180 μL of Assay Buffer, then 0.005% sample was made by mixing 20 μL of 0.05% sample with 180 μL of Assay Buffer.

### Sample Binding Step.

Catch-0 plates prepared by immobilizing the aptamer mixes on the Streptavidin Magnetic Sepharose beads as described above. Frozen plates were thawed for 30 min at 25° C. and were washed once with 175 μL of Assay Buffer. 100 μL of each sample dilution (20%, 0.5% and 0.005%) were added to the plates containing beads with three different aptamer master mixes (Dil1, Dil2 and Dil3, respectively). Catch-0 plates were then sealed with aluminum foil seals (Microseal ‘F’ Foil, Bio-Rad) and placed in the 4-plate rotating shakers (PHMP-4, Grant Bio) set at 850 rpm, 28° C. Sample binding step was performed for 3.5 hours.

### Multiplex Aptamer Assay Processing on Fluent Robot.

After sample binding step was completed, Catch-0 plates were placed into aluminum plate adapters and placed on the robot deck. Magnetic bead wash steps were performed using a temperature-controlled plate. For all robotic processing steps, the plates were set at 25° C. temperature except for Catch-2 washes as described below. Plates were washed 4 times with 175 μL of Assay Buffer, each wash cycle was programmed to shake the plates at 1000 rpm for at least 1 min followed by separation of the magnetic beads for at least 30 seconds before buffer aspiration. During the last wash cycle, the Tag reagent was prepared by diluting 100×Tag reagent (EZ-Link NHS-PEG₄-Biotin, part number 21363, Thermo, 100 mM solution prepared in anhydrous DMSO) 1:100 in the Assay buffer and poured in the trough on the robot deck. 100 μL of Tag reagent was added to each of the wells in the plates and incubated with shaking at 1200 rpm for 5 min to biotinylate proteins captured on the bead surface. Biotinylation reactions were quenched by addition of 175 μL of Quench buffer (20 mM glycine in Assay buffer) to each well. Plates were incubated static for 3 min then washed 4 times with 175 μL of Assay buffer, washes were performed under the same conditions as described above.

### Photo-Cleavage and Kinetic Challenge.

After the last wash of the plates, 90 μL of Photocleavage buffer (2 μM of a oligonucleotide competitor in Assay buffer; the competitor has the nucleotide sequence of 5′-(AC-Bn-Bn)₇-AC-3′, where Bn indicates a 5-position benzyl-substituted deoxyuridine residue) was added to each well of the plates. The plates were moved to a photocleavage substation on the Fluent deck. The substation consists of the BlackRay light source (UVP XX-Series Bench Lamps, 365 nm) and three Bioshake 3000-T shakers (Q Instruments). Plates were irradiated for 20 min minutes with shaking at 1000 rpm.

### Catch-2 Bead Capture.

At the end of the photocleavage process, the buffer was removed from Catch-2 plate via magnetic separation, plate was washed once with 100 μL of Assay buffer. Photo-cleaved eluate containing aptamer-protein complexes was removed from each Catch-0 plate starting with the dilution 3 plate. All 90 μL of the solution was first transferred to the Catch-1 Eluate plate positioned on the shaker with raised magnets to trap any Steptavidin Magnetic Sepharose beads which might have been aspirated. After that, solution was transferred to the Catch-2 plate and the plate was incubated for 3 min with shaking at 1400 rpm at 25° C. After the incubation for 3 min, the magnetic beads were separated for 90 seconds, solution removed from the plate and photocleaved Dil2 plate solution was added to plate. Following identical process, the solution from Dil1 plate was added and incubated for 3 min. At the end of the 3 min incubation, 6 μL of the MB Block buffer was added to the magnetic bead suspension and beads were incubated for 2 min with shaking at 1200 rpm at 25° C. After this incubation, the plate was transferred to a different shaker which was preset to 38° C. temperature. Magnetic beads were separated for 2 minutes before removing the solution. Then, the Catch-2 plate was washed 4 times with 175 μL of MB Wash buffer (20% glycerol in Assay Buffer), each wash cycle was programmed to shake the beads at 1200 rpm for 1 min and allow the beads to partition on the magnet for 3.5 minutes. During the last bead separation step, the shaker temperature was set to 25° C. Then beads were washed once with 175 μL of Assay buffer. For this wash step, beads were shaken at 1200 rpm for 1 min and then allowed to separate on the magnet for 2 minutes. Following the wash step, aptamers were eluted from the purified aptamer-protein complexes using Elution buffer (1.8 M NaCl₄, 40 mM PIPES, pH 6.8, 1 mM EDTA, 0.05% Triton X-100). Elution was done using 75 μL of Elution buffer for 10 min at 25° C. shaking beads at 1250 rpm. 70 μL of the eluate was transferred to the Archive plate and separated on the magnet to partition any magnetic beads which might have been aspirated. 10 μL of the eluted material was transferred to the black half-area plate, diluted 1:5 in the Assay buffer and used to measure the Cy3 fluorescence signals which are monitored as internal assay QC. 20 μL of the eluted material was transferred to the plate containing 5 μL of the Hybridization Blocking solution (Oligo aCGH/ChIP-on-chip Hybridization Kit, Large Volume, Agilent Technologies 5188-5380, containing a spike of Cyanine 3-labeled DNA sequence complementary to the corner marker probes on Agilent arrays). This plate was removed from the robot deck and further processed for hybridization (see below). Archive plate with the remaining eluted solution was heat-sealed using aluminum foil and stored at −20° C.

### Hybridization.

25 μL of 2× Agilent Hybridization buffer (Oligo aCGH/ChIP-on-chip Hybridization Kit, Agilent Technologies, part number 5188-5380) was manually pipetted to the each well of the plate containing the eluted samples and blocking buffer. 40 μL of this solution was manually pipetted into each “well” of the hybridization gasket slide (Hybridization Gasket Slide—8 microarrays per slide format, Agilent Technologies). Custom SurePrint G3 8×60k Agilent microarray slides containing 10 probes per array complementary to each aptamer were placed onto the gasket slides according to the manufacturer's protocol. Each assembly (Hybridization Chamber Kit—SureHyb enabled, Agilent Technologies) was tightly clamped and loaded into a hybridization oven for 19 hours at 55° C. rotating at 20 rpm.

### Post-Hybridization Washing.

Slide washing was performed using Little Dipper Processor (model 650C, Scigene). Approximately 700 mL of Wash Buffer 1 (Oligo aCGH/ChIP-on-chip Wash Buffer 1, Agilent Technologies) was poured into large glass staining dish and used to separate microarray slides from the gasket slides. Once disassembled, the slides were quickly transferred into a slide rack in a bath containing Wash Buffer 1 on the Little Dipper. The slides were washed for five minutes in Wash Buffer 1 with mixing via magnetic stir bar. The slide rack was then transferred to the bath with 37° C. Wash Buffer 2 (Oligo aCGH/ChIP-onchip Wash Buffer 2, Agilent Technologies) and allowed to incubate for five minutes with stirring. The slide rack was slowly removed from the second bath and then transferred to a bath containing acetonitrile and incubated for five minutes with stirring.

### Microarray Imaging.

The microarray slides were imaged with a microarray scanner (Agilent G4900DA Microarray Scanner System, Agilent Technologies) in the Cyanine 3-channel at 3 μm resolution at 100% PMT setting and the 20-bit option enabled. The resulting tiff images were processed using Agilent Feature Extraction software (version 10.7.3.1 or higher) with the GE1_1200_Jun14 protocol.

1. A method comprising:
a) measuring the level of Sonic Hedgehog (SHH) protein, and the level of at least one, two, three, four, five, six, seven, eight, nine, ten, eleven, twelve or thirteen proteins selected from the group consisting of PGAM1, PGAM2, C4A.C4B, PTPN4, TNFSF14, FAM49B, RBP7, IHH, DDX39B, S100A12, IL21R, TMEM9 and ADAM9 in a sample from a human subject; and
b) identifying the sample as an analysis sample or negative sample based on the level of SHH and the level of the one, two, three, four, five, six, seven, eight, nine, ten, eleven, twelve or thirteen proteins;
wherein, the analysis sample is a sample that is used in one or more of the following: protein biomarker discovery analysis, protein expression level analysis, a diagnostic method or a prognostic method, and the negative sample is a sample that is not used as an analysis sample.

2. The method of claim 1, wherein the measuring is performed using mass spectrometry, an aptamer based assay and/or an antibody based assay.

3. The method of claim 1, wherein the sample is selected from blood, plasma, serum or urine.

4. The method of claim 1, wherein the method comprises measuring SHH and PGAM1, SHH and PTPN4, SHH and TNFSF14, SHH and FAM49B, SHH and RBP7, SHH and IHH, SHH and DDX39B, SHH and S100A12, SHH and PGAM2, SHH and C4A.C4B, SHH and IL21R, SHH and TMEM9 or SHH and ADAM9.

5. The method of claim 1, wherein the method comprises measuring SHH, PGAM1 and TNFSF14; SHH, PGAM1 and RBB7; SHH, PGAM1 and PTPN4; SHH, PGAM1 and DDX39B; SHH, PGAM1 and FAM49B; SHH, PGAM1 and IHH; SHH, PGAM1 and S100A12; SHH, PGAM1 and ADAM9; SHH, PTPN4 and RBP7; SHH, PTPN4 and TNFSF14; SHH, PTPN4 and IHH; SHH, RBP7 and FAM49B; SHH, RBP7 AND IHH; SHH, FAM49B and TNFSF14; SHH, DDX39B and PTPN4; SHH, TNFSF14 and S100A12; SHH, IHH and RBP7; SHH, IHH and TNFSF14; SHH RBP7 and TNFSF14; SHH, RBP7 and S100A12; SHH, RBP7 and DDX39B; SHH, TNFSF14 and DDX39B; SHH, S100A12 and DDX39B; SHH, FAM49B and S100A12; SHH, IHH and FAM49B; SHH, IHH and DDX39B; SHH, TNF and ADAM9; SHH FAM49B and DDX39B; SHH, IHH and ADAM9; SHH, PGAM1 and C4A.C4B; SHH, PGAM2 and RBP7; SHH, PGAM1 and IL21R; SHH, PGAM2 and PTPN4; SHH, PGAM2 and ADAM9, SHH, PGAM2 and C4A.C4B; SHH, PGAM2 and IL21R; SHH, IHH and PGAM2; SHH, PGAM1 and PGAM2; SHH, TMEM9 and PGAM2 or SHH, TMEM9 and PGAM1.

6. The method of claim 1, wherein the method comprises measuring SHH and PGAM1, and at least two of the following proteins selected from RBP7, TNFSF14, PTPN4, DDX39B, FAM49B, S100A12, IHH, PGAM2, C4A.C4B, IL21R, TMEM9 and ADAM9.

7. The method of claim 1, wherein the method comprises measuring SHH and IHH, and at least two of the following proteins selected from RBP7, TNFSF14, PTPN4, DDX39B, FAM49B, S100A12, PGAM1, PGAM2, C4A.C4B, IL21R, TMEM9 and ADAM9.

8. The method of claim 1, wherein the protein levels are used to predict the length of time between the sample collection from the human subject and sample centrifugation and/or the length of time between sample centrifugation and sample decanting.

9. The method of claim 8, wherein the time between sample collection and sample centrifugation is about from 0 hours to 0.5 hours; 0.5 hours to 1.5 hours; 1.5 hours to 3 hours; 3 hours to 9 hours; 9 hours to 24 hours or greater than 24 hours, and/or the time between sample centrifugation and sample decanting is about from 0 hours to 0.5 hours; 0.5 hours to 1.5 hours; 1.5 hours to 3 hours; 3 hours to 9 hours; 9 hours to 24 hours or greater than 24 hours.

10-112. (canceled)
